(19) United States
(12) Patent Application Publication
(10) Pub. No.: US 2025/0003883 A1
(43) Pub. Date: Jan. 2, 2025

(54) SERS NANOSENSOR FOR DETECTING SUBSTANCES PRODUCED IN PLANTS, MANUFACTURING METHOD THEREOF, AND PLANT MONITORING DEVICE USING SERS NANOSENSOR AND METHOD

(71) Applicant: SEOUL NATIONAL UNIVERSITY R&DB FOUNDATION, Seoul (KR)

(72) Inventors: Dae Hong Jeong, Seoul (KR); Seonyeong Kwak, Seoul (KR); Won Ki Son, Seoul (KR); Dong Uk Shin, Seoul (KR)

(21) Appl. No.: 18/705,360

(22) PCT Filed: Oct. 28, 2022
(86) PCT No.: PCT/KR2022/016695

§ 371 Date: Apr. 26, 2024

(30) Foreign Application Priority Data
Oct. 29, 2021 (KR) 10-2021-0146340
Oct. 27, 2022 (KR) 10-2022-0140236

(51) Int. Cl. G01N 21/65; G01N 33/00

(52) U.S. Cl. CPC G01N 21/658; G01N 33/0098

(57) ABSTRACT

The present disclosure provides a surface-enhanced Raman scattering (SERS) nanosensor for detecting a substance produced within a plant including a first nanostructure, a second nanostructure containing a metal and disposed on a surface of the first nanostructure to cause SERS, and a polymer material bound to a surface of the second nanostructure, and generating an attraction force that attracts the substance produced within the plant.

### TECHNICAL FIELD

The present invention relates to sensors for detecting substances and their manufacture and use, and more particularly, to nanosensors for detecting substances produced in living organisms and their manufacture and use.

### BACKGROUND ART

Use of chemical fertilizers and pesticides, which were the driving force behind the increase in crop production, led to soil and groundwater contamination, and as a result of it, leads to a paradox of the green revolution in which food production scale decreased. As production growth has decreased, the need for sustainable agriculture has emerged, and precision agriculture which may maximize production per unit area while continuing to coexist with the environment, has emerged as a measure to overcome the immediate crisis.

Modern agriculture is recognized as a new innovative growth engine through the integration of cutting-edge science and technology. Recently, the development of ICBM (IoT, Cloud, Big Data, Mobile) and AI (artificial intelligence) technologies and the popularization of network use, coupled with the expansion of investment in agricultural technology, are escaping the economic and technological constraints of precision agriculture. The global smart farm market size was $196 billion in 2016, and is expected to grow at an average annual rate of about 16.4%, reaching about $408 billion by 2020. Major developed countries such as the United States and Europe recognize agriculture as a future growth industry and are seeking new opportunities in the agricultural field, led by global IT (information technology) companies such as Google. As global fund investments in Agtech (agriculture technology) venture companies amount to approximately $10.2 billion, mainly in the U.S. and Europe, expectations and an investment environment for precision agriculture are being created.

Due to the effects of global warming, the need to strengthen risk factor management is emerging due to not only the increase in existing pests but also the continuous emergence of new pests. It is estimated that up to 50% of global harvests of key food resources will be lost because of rapid increases in the metabolic and reproductive rates of pests due to climate change. Recently, foreign pests have spread rapidly across the country in Korea, and since 2000, the number of plant diseases introduced into the country has reached 21 (Rural Development Administration, 2017 Crop Pest Surveillance Report), and many of them have already reached the ‘regular occurrence’ stage.

Meanwhile, as income increases, the level of demand for quality of life is increasing, and among these, consumers' expectations regarding food safety are increasing. However, as trade in agricultural products between countries continues to increase, the risk that foreign plant pathogens are introduced into the country is increasing. There is a global trend to improve food safety, enact food safety laws, and prepare guidelines to respond to the demand for safe food and the risks posed by the global trade system. The United States is raising food safety standards and strengthening imported food safety management standards with a focus on prevention by revising the Food Safety Modernization Act (FSMA) in 2011. China sought to strengthen the safety of Chinese agricultural products and improve the safety management standards for imported agricultural products through the revision of the Food Safety Act in 2015. Korea has been implementing the Positive List System (PLS), which strengthens pesticide residue tolerance standards since 2019 in order to ensure safe consumption for consumers of agricultural products.

As a measure to improve crop yield per unit area within limited resources and prevent side effects from indiscriminate control, early diagnosis of crop pests and diseases and crop health monitoring technology are emerging as alternatives. Rapid and accurate early diagnosis of plant diseases may be a measure to minimize economic damage caused by the spread of diseases, prevent overuse of pesticides, and solve safety problems caused by pesticide residues.

For the early diagnosis of plant diseases, efforts are required to apply nano biotechnology, which is actively applied to early diagnosis and treatment of diseases in the bio and medical fields, to the agricultural field. Since nanomaterials may overcome the limitations of existing materials by using new properties formed at the nanometer size, and are being used throughout the 6T (technology) industry as a foundational technology for future industries. The impact of nanotechnology combined with agricultural science is expected to surpass agricultural mechanization and the green revolution, which formed the basis of modern agricultural development.

Early diagnosis of plant diseases or real-time monitoring of plant health status will be an important technology in precision agriculture in that it may prevent the spread of infectious diseases, prevent side effects of overuse of preventive drugs, and improve crop production and safety. However, in the diagnosis of plant diseases and monitoring of plant health, there are technological demands that non-destructive and real-time measurement must be possible and implemented in a simple manner, early diagnosis must be possible even at very low concentrations of the target substance, and continuous monitoring of various target substances must be possible.

### DISCLOSURE OF THE INVENTION

### Technical Problem

The technological object to be achieved by the present invention is to provide a SERS (surface-enhanced Raman scattering) nanosensor which may be usefully applied to the diagnosis of plant diseases or monitoring of plant conditions, and may easily detect substances produced within a plant (substances produced in a plant).

In addition, the technological object to be achieved by the present invention is to provide a manufacturing method of the SERS nanosensor.

In addition, the technological object to be achieved by the present invention is to provide a plant monitoring apparatus and method using the SERS nanosensor.

The objects to be solved by the present invention is not limited to the objects mentioned above, and other objects not mentioned will be understood by those skilled in the art from the description below.

### Technical Solution

According to one embodiment of the present invention, there is a provided a surface-enhanced Raman scattering (SERS) nanosensor for detecting a substance produced within a plant comprising: a first nanostructure; a second nanostructure containing a metal and disposed on a surface of the first nanostructure to cause SERS; and a polymer material bound to a surface of the second nanostructure and generating an attraction force that attracts the substance produced within the plant.

The first nanostructure may include a non-metal.

The first nanostructure may have a shape of a nanoparticle or a nanotube.

The first nanostructure may include a silica or a carbon nanotube (CNT).

The second nanostructure may include a plurality of nanoparticles.

The second nanostructure may include at least one of Ag and Au.

The first nanostructure may include a silica nanoparticle, the second nanostructure may include a plurality of Ag nanoparticles disposed on a surface of the silica nanoparticle, the silica nanoparticle may constitute a core portion and the plurality of Ag nanoparticles may constitute a shell portion.

The first nanostructure may include a CNT, and the second nanostructure may include a plurality of Au nanoparticles disposed on a surface of the CNT.

The polymer material may include PDDA [poly(diallyldimethylammonium chloride)].

The substance produced within the plant may include a plant hormone molecule generated by plant stress or disease.

The substance produced within the plant may include at least one of phytoalexin, salicylic acid (SA), adenosine triphosphate (ATP), indole-3-acetic acid (IAA), folic acid (FA), thiamine, and nasturlexin.

According to another embodiment of the present invention, a SERS nanosensor for detecting a substance produced within a plant as described above; and a plant monitoring apparatus including a Raman spectrometer for detecting a SERS signal generated from the SERS nanosensor are provided.

According to another embodiment of the present invention, there is provided a plant monitoring method comprising: introducing the above-described SERS nanosensor for detecting a substance produced in a plant into a plant (plant body); and measuring a SERS signal generated from the SERS nanosensor by using Raman spectroscopy.

According to another embodiment of the present invention, there is provided a manufacturing method of a surface-enhanced Raman scattering (SERS) nanosensor for detecting a substance produced within a plant comprising: preparing a first nanostructure; forming a second nanostructure which is disposed on a surface of the first nanostructure, contains a metal and causes SERS; and binding a polymer material which generates an attraction force that attracts the substance produced within the plant to a surface of the second nanostructure,

The first nanostructure may include a silica nanoparticle, the second nanostructure may include a plurality of Ag nanoparticles disposed on a surface of the silica nanoparticle, the silica nanoparticle may constitute a core portion, and the plurality of Ag nanoparticles may constitute a shell portion.

The manufacturing method of the SERS nanosensor for detecting substances produced in plants may include functionalizing the surface of the silica nanoparticle with a thiol group by using 3-mercaptopropyltrimethoxysilane; forming the plurality of Ag nanoparticles on the surface of the silica nanoparticle by using hexadecylamine and silver nitrate; and functionalizing the surface of the plurality of Ag nanoparticles with the polymer material.

The first nanostructure may include a carbon nanotube (CNT), and the second nanostructure may include a plurality of Au nanoparticles disposed on a surface of the CNT.

The polymer material may include PDDA [poly(diallyldimethylammonium chloride)].

### Advantageous Effects

According to embodiments of the present invention, a SERS nanosensor which may be usefully applied to the diagnosis of plant diseases or monitoring plant conditions, and may easily detect substances produced within a plant (substances produced in a plant) may be implemented. Additionally, according to embodiments of the present invention, a plant monitoring apparatus and method using the SERS nanosensor may be implemented.

According to embodiments of the present invention, core technological capabilities for early diagnosis of plant diseases may be secured through the convergence of nanotechnology (NT) and biotechnology (BT). All technologies and platforms related to nanosensors according to embodiments of the present invention may be used to develop nanosensors (nano-optical sensors) for early diagnosis of various crop diseases. The plant diagnosis technology using the nanosensor described above may be employed usefully in developing reliable disease response measures through accurate and rapid initial diagnosis before lesions occur in actual agricultural industry sites.

In addition, early diagnosis of plant diseases using the above-mentioned nanosensor is a simple, non-destructive method which is easy to apply to various plant species, and may be a real-time detection method which may detect signals immediately after introducing the nanosensor into the plant. Therefore, it is expected that the early diagnosis of plant diseases (i.e., plant monitoring method) using the nanosensor according to the embodiment may be commercialized as a type of a platform by considering user convenience.

Additionally, embodiments of the present invention may be applied to precision agriculture, smart agriculture, new crop screening technology, plant biotechnology-based pharmaceutical production, etc.

The expected effects of the technology according to the embodiments of the present invention may be summarized from technological and economic and industrial aspects as follows.

### Expected Effects From A Technological Aspect

\textcircled{1} Advancement of early diagnosis method development technology for plant diseases and expansion of research fields.

\textcircled{2} Securing source technology for early diagnosis of plant diseases using nanotechnology.

\textcircled{3} Contribution to standardizing early and precise diagnosis of plant fungal diseases using nanosensors.

\textcircled{4} Providing new and more efficient technologies for producing organic products.

### Expected Effects in Economic and Industrial Aspects

\textcircled{1} Through early diagnosis using nanosensors, damage to farms is reduced by preventing and alleviating crop diseases which are problematic not only during the growing period but also after harvest and for which pesticides are less effective.

\textcircled{2} Expect the development of related industries through popularization of plant disease diagnosis technology using nanosensors and vitalization of research.

\textcircled{3} Revitalizing the domestic eco-friendly agricultural industry and promoting agricultural exports.

However, the effects of the present invention are not limited to the above effects and may be expanded in various ways without departing from the technological spirit and scope of the present invention.

### BRIEF DESCRIPTION OF THE DRAWINGS

FIG. 1 is a cross-sectional diagram illustrating a surface-enhanced Raman scattering (SERS) nanosensor for detecting a substance produced within a plant, according to an embodiment of the present invention.

FIG. 2 is a diagram for explaining a SERS nanosensor for detecting a substance produced within a plant and a manufacturing method thereof according to a specific embodiment of the present invention.

FIG. 3 is a conceptual diagram illustrating enhanced Raman scattering occurring on the surface of a SERS nanosensor according to an embodiment of the present invention.

FIG. 4 is a TEM (transmission electron microscope) image of a SERS nanosensor synthesized according to an embodiment of the present invention.

FIG. 5 is a scanning electron microscope (SEM) image of a SERS nanosensor synthesized according to an embodiment of the present invention.

FIG. 6 is a graph illustrating the results of evaluating the hydrodynamic diameter of the SERS nanosensor synthesized according to an embodiment of the present invention and the structure according to the comparative example.

FIG. 7 is a graph illustrating a comparison of the UV-visible extinction spectrum of a SERS nanosensor synthesized according to an embodiment of the present invention and a structure according to a comparative example.

FIG. 8 is a graph illustrating a comparison of the Raman enhancement factors of a SERS nanosensor (i.e., AgNS@PDDA) synthesized according to an embodiment of the present invention and a structure (i.e., AgNS) according to a comparative example.

FIG. 9 is a graph illustrating a comparison of the zeta potential of a SERS nanosensor (i.e., AgNS@PDDA) synthesized according to an embodiment of the present invention and a structure (i.e., AgNS) according to a comparative example.

FIG. 10 is a graph illustrating in vitro SERS spectra of AgNS, AgNS to which 1 μM ATP (adenosine triphosphate) was applied, AgNS@PDDA, and AgNS@PDDA to which 1 μM ATP was applied.

FIG. 11 is a graph illustrating the change in zeta potential (black) and the change in SERS intensity (magenta) at 729 cm⁻¹ of AgNS@PDDA according to ATP concentration.

FIG. 12 is a graph illustrating the change in zeta potential of the nanosensor for the change in normalized SERS signal intensity of ATP in case of addition or removal of 1 μM ATP.

FIG. 13 is a graph illustrating the change in the SERS spectrum of the nanosensor depending on the presence or absence of 1 μM ATP.

FIG. 14 is a schematic diagram illustrating infiltration of AgNS@PDDA nanosensors through stomatal pores of a plant and distribution of nanosensors in the cross section of plant leaves.

FIG. 15 shows CLSM (confocal laser scanning microscopy) images of watercress, barley, and wheat leaves into which dye-labeled silica nanoparticles (diameter 300 nm, 0.1 mg/mL) were introduced.

FIG. 16 is a diagram illustrating an overlay of brightfield images and confocal SERS intensity maps of the epidermis and mesophyll of watercress, barley, and wheat leaves.

FIG. 17 is a graph illustrating the SERS spectrum of plant leaves infiltrated with AgNS@PDDA nanosensors.

FIG. 18 is a diagram illustrating the confocal SERS intensity map of barley leaves infiltrated with AgNS@PDDA (0.1 mg/mL) and the spectra of selected spots with different SERS intensities.

FIG. 19 is a graph illustrating SERS spectra of various plant hormone molecules.

FIG. 20 and FIG. 21 are graphs illustrating the concentration dependence of SERS intensity in binary mixtures.

FIG. 22 and FIG. 23 are graphs illustrating 3D plots of the SERS bands of SA and ATP according to their concentrations in a mixture of SA and ATP.

FIG. 24 is a diagram for explaining a plant monitoring apparatus using a SERS nanosensor according to an embodiment of the present invention and a plant monitoring method.

FIG. 25 is a photographic image illustrating a leaf of watercress into which a SERS nanosensor was introduced according to an embodiment of the present invention.

FIG. 26 is a graph illustrating the results of detecting the SERS signal generated by substances (molecules) generated by wound stimulation for watercress into which a SERS nanosensor was introduced.

FIG. 27 is a graph illustrating the results of measuring changes in Raman signals after fungal infection in wheat and barley according to an embodiment of the present invention.

FIG. 28 is a graph illustrating the results of measuring detection frequency of plant hormones obtained from wheat and barley over time after fungal infection in wheat and barley according to an embodiment of the present invention.

FIG. 29 is a diagram for explaining detection of SERS signals in living plants subjected to abiotic stress such as cold or wound.

FIG. 30 and FIG. 31 are diagrams illustrating brightfield images of a small area of a leaf where SERS area scanning was performed.

FIG. 32 to FIG. 34 are graphs illustrating Raman spectra obtained in region A, region B, and region C, respectively, at specific time intervals after wounding the leaf.

FIG. 35 and FIG. 36 are graphs illustrating the temporal profile of the SERS band at 1353 cm⁻¹ or 729 cm⁻¹ related to cruciferous phytoalexin or eATP obtained from the three regions A, B and C described above.

FIG. 37 is a graph illustrating the SERS spectrum obtained from in vitro 1 mM GSH (green), a leaf (blue) infiltrated with 50 μM GSH, a leaf (red) of plants after chilling stress for 24 hours at 4° C., and a control plant leaf (grown under normal conditions, black).

FIG. 38 is a graph illustrating signal comparison between control watercress plants and plants under wounding conditions.

FIG. 39 is a graph illustrating signal comparison between control watercress plants and plants under cold stress conditions.

FIG. 40 is a diagram schematically illustrating a SERS-based monitoring method for signaling molecules in living crops infected with fungi.

FIG. 41 is a graph illustrating the SERS spectrum obtained from barley leaves in order to confirm the 1035 cm⁻¹ band due to SA.

FIG. 42 and FIG. 43 are graphs illustrating representative SERS spectra obtained while monitoring the progression of fungal disease in infected barley and wheat plants, respectively.

FIG. 44 is a photographic image illustrating lesion formation caused by fungal pathogens on barley and wheat leaves.

FIG. 45 is a graph illustrating the results of real-time PCR (polymerase chain reaction) analysis of fungal DNA content during disease progression.

FIG. 46 shows a SERS intensity map obtained from infected wheat plants on day 2.

FIG. 47 and FIG. 48 are graphs illustrating representative histograms for presumptive concentrations of SA (red) and ATP (green) in living barley and wheat plants infected with F. graminearum, respectively.

FIG. 49 and FIG. 50 are graphs obtained by comparing the signals between control crop plants and plants under fungal infection condition.

FIG. 51 and FIG. 52 are graphs illustrating the expression of ICS1 (isochorismate synthase), PAL (phenylalanine ammonia lyase), and selected PR (pathogenesis-related) genes induced in barley and wheat leaves inoculated with F. graminearum.

FIG. 53 is a diagram illustrating a SERS nanosensor for detecting a substance produced in a plant, according to another embodiment of the present invention.

FIG. 54 is a graph illustrating a SERS spectrum showing a characteristic of simultaneously detecting ATP and thiamine by using the SERS nanosensor according to the example of FIG. 53.

FIG. 55 is a SERS intensity map illustrating the results of detecting a signal caused by a wound in a watercress plant by using the SERS nanosensor according to the embodiment of FIG. 53.

FIG. 56 is a graph illustrating raw SERS spectra for multiple molecular signals obtained at specific points of the SERS intensity map of FIG. 55.

### BEST MODE FOR CARRYING OUT THE INVENTION

Hereinafter, embodiments of the present invention will be described in detail with reference to the accompanying drawings.

The embodiments of the present invention to be described below are provided to more clearly explain the present invention to those skilled in the art, and the scope of the present invention is not limited by the following embodiments, and the embodiments may be modified in many different forms.

The terms used in this specification are used to describe specific embodiments and are not intended to limit the present invention. The terms indicating a singular form used herein may include plural forms unless the context clearly indicates otherwise. Also, as used herein, the terms, “comprise” and/or “comprising” specify the presence of the stated shape, step, number, operation, member, element, and/or group thereof and does not exclude the presence or addition of one or more other shapes, steps, numbers, operations, elements, elements and/or groups thereof. In addition, the term, “connection” used in this specification means not only a direct connection of certain members, but also a concept including an indirect connection in which other members are interposed between the members.

In addition, in the present specification, when a member is said to be located “on” another member, this arrangement includes not only a case in which a member is in contact with another member, but also a case where another member exists between the two members. As used herein, the term, “and/or” includes any one and all combinations of one or more of the listed items. In addition, the terms of degree such as “about” and “substantially” used in the present specification are used as a range of values or degrees, or as a meaning close thereto, taking into account inherent manufacturing and substance tolerances, and exact or absolute numbers provided to aid in the understanding of this application are used to prevent the infringers from unfairly exploiting the stated disclosure.

Hereinafter, embodiments of the present invention will be described in detail with reference to the accompanying drawings. A size or a thickness of areas or parts shown in the accompanying drawings may be slightly exaggerated for clarity of the specification and convenience of description. The same reference numbers indicate the same configuring elements throughout the detailed description.

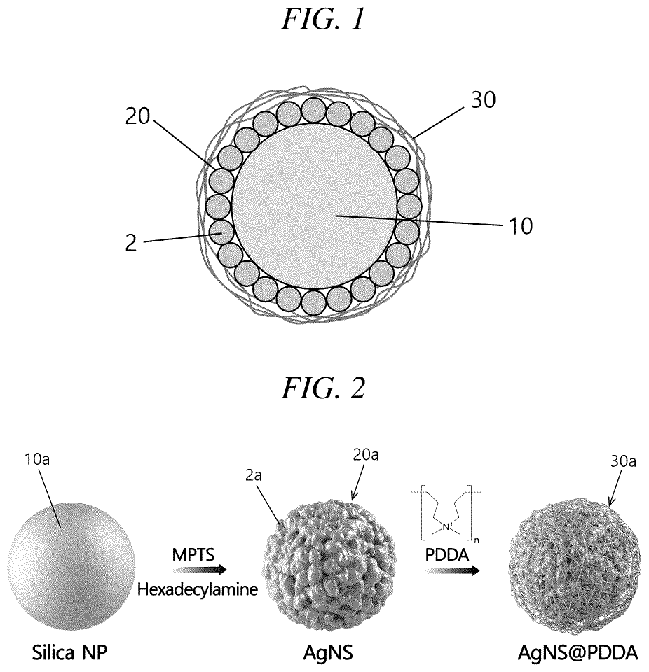

FIG. 1 is a cross-sectional diagram illustrating a surface-enhanced Raman scattering (SERS) nanosensor for detecting a substance produced within a plant, according to an embodiment of the present invention.

Referring to FIG. 1, the SERS nanosensor according to an embodiment of the present invention may be an optical nanosensor for detecting a substance produced within a plant (a substance produced in a plant). The SERS nanosensor may include a first nanostructure 10, a second nanostructure 20 which is placed on the surface of the first nanostructure 10 to cause (induce) SERS and contains a metal, and a polymer material 30 which is bound to the surface of 20 and generate an attraction force that attracts the substance produced within the plant.

The first nanostructure 10 may include or be composed of a non-metal. In this embodiment, the first nanostructure 10 may be a dielectric (an insulator). As a specific example, the first nanostructure 10 may include or be composed of a silica. Additionally, in this embodiment, the first nanostructure 10 may have a nanoparticle shape. At this time, the diameter of the first nanostructure 10 may be several tens of nm to several hundreds of nm. For example, the diameter of the first nanostructure 10 may be about 50 nm or more and less than about 1000 nm. However, the form of the first nanostructure 10 is not limited to a nanoparticle shape. The first nanostructure 10 may have another shape such as a nanotube. The first nanostructure 10 may serve as a base for forming the second nanostructure 20 on its surface. Additionally, the first nanostructure 10 may be bonded to the second nanostructure 20 to play a role in enhancing the plasmon effect.

The second nanostructure 20 may include a plurality of nanoparticles 2. The nanoparticle 2 may be an element for causing (inducing) SERS for the substance produced within the plant. The nanoparticle 2 may include or be composed of metals such as Ag and Au. For example, the nanoparticle 2 may be an Ag nanoparticle. The nanoparticle 2 may have a smaller size than the first nanostructure 10. The diameter of the nanoparticle 2 may be hundreds of nm or less or tens of nm or less. For example, the diameter of the nanoparticle 2 may be about 1 nm or more and about 300 nm or less. However, this diameter range of nanoparticle 2 is exemplary and may vary depending on the case. The plurality of nanoparticles 2 may form one bumpy nanoshell on the surface of the first nanostructure 10. In this case, the second nanostructure 20 may be said to have a nanoshell structure. The outer diameter of the nanoshell (a bumpy nanoshell) may be, for example, about 50 nm or more and less than about 1000 nm. The second nanostructure 20 may serve to amplify SERS enhancement.

According to one embodiment, the first nanostructure 10 may be a silica nanoparticle, and the second nanostructure 20 may be composed of a plurality of Ag nanoparticles disposed on the surface of the silica nanoparticle. At this time, the silica nanoparticle may constitute a core portion, and the plurality of Ag nanoparticles may constitute a shell portion. Therefore, it may be said that the first nanostructure 10 and the second nanostructure 20 constitute one core-shell structure. The shell part may be called as Ag bumpy nanoshell. The Ag bumpy nanoshell may play a role in amplifying SERS enhancement to about 10⁷ or higher.

The polymer material 30 may be formed on the surface of the second nanostructure 20 and may serve to generate an attraction force that attracts the substance produced within the plant. It may be said that the surface of the second nanostructure 20 is functionalized with the polymer material 30. The polymer material 30 may have a predetermined positive (+) charge. This polymer material 30 may pull a negatively charged substances produced within the plant and allow them to contact or be disposed close to the surface of the second nanostructure 20. When a predetermined laser is irradiated to the second nanostructure 20 which induces surface plasmon, excitation of an energy state occurs, and at this time, a strong electromagnetic field may be formed within a certain range from the second nanostructure 20. The Raman intensity by SERS of the substance (molecule) produced in the plant placed in contact with or close to the second nanostructure 20 may remarkably increase due to the electromagnetic field. Therefore, the SERS nanosensor according to the embodiment may be usefully used to detect substances (molecules) produced within the plant.

The polymer material 30 may be, for example, PDDA [poly(diallyldimethylammonium chloride)] or may include it. When PDDA is applied as a polymer material 30, PDDA may effectively perform a role to attract (pull) substances produced within the plant, and as a result, the detection characteristics of substances produced within the plant using SERS may be greatly improved. However, the polymer material 30 is not limited to PDDA and may change depending on the case. The substance produced within the plant may include plant hormone molecules generated by plant stress or disease. The plant hormone molecule may be a small molecule. Plants may produce certain plant hormone molecules as a result of an immune response due to stress or disease. In an embodiment of the present invention, the plant's health status or disease occurrence may be easily monitored by detecting the plant hormone molecules through use of a SERS nanosensor.

The substance produced within the plant may include, for example, at least one of phytoalexin, salicylic acid (SA), adenosine triphosphate (ATP), indole-3-acetic acid (IAA), folic acid (FA), thiamine, and nasturlexin. These may be small molecules corresponding to plant hormones. However, the substances produced within the plant which are targets of detection are not limited to the above descriptions, and may further include other hormone substances.

The diameter or thickness of the SERS nanosensor according to an embodiment of the present invention may be, for example, about 20 nm or more and less than about 1000 nm. The length of the SERS nanosensor may be tens of nm or more, and in some cases, may be several um. However, the dimensional range of these SERS nanosensors is exemplary and may vary depending on the case. Since the SERS nanosensor according to the embodiment may utilize the plasmon effect, it may also be referred to as a “plasmonic nanosensor.”

FIG. 2 is a diagram for explaining a SERS nanosensor for detecting a substance produced within a plant and a manufacturing method thereof according to a specific embodiment of the present invention.

Referring to FIG. 2, the manufacturing method of a SERS nanosensor for detecting a substance produced in a plant according to an embodiment of the present invention may include a step for preparing a first nanostructure 10a, a step for forming a second nanostructure 20a which is disposed on a surface of the first nanostructure 10a, contains a metal and causes SERS, and a step for combining a polymer material 30 which generates an attraction force that attracts the substance produced in the plant toward a surface of the second nanostructure 20a.

According to one example, the first nanostructure 10a may be a silica nanoparticle. The second nanostructure 20a may be composed of a plurality of Ag nanoparticles disposed on the surface of the silica nanoparticles. In this case, the silica nanoparticle may constitute a core portion, and the plurality of Ag nanoparticles may constitute a shell portion. The shell portion may be Ag bumpy nanoshell (i.e., AgNS).

More specifically, the method for manufacturing the SERS nanosensor may include a step for functionalizing the surface of the silica nanoparticle with a thiol group (i.e. —SH group) by using 3-mercaptopropyltrimethoxysilane (i.e. MPTS), a step for forming the plurality of Ag nanoparticles by using hexadecylamine and silver nitrate on the surface of the silica nanoparticle and a step for functionalizing the surface of the plurality of Ag nanoparticles with the polymer material. At this time, the polymer material may be PDDA.

As a first step, the silica nanoparticle (i.e., a silica dielectric core) may be synthesized according to the Stober method. 1.6 mL of TEOS (tetraethyl orthosilicate) may be dissolved in 40 mL of EtOH and 3.5 mL of ammonium hydroxide solution may be added. After vigorously stirring this reaction mixture for 20 hours, the silica nanoparticles, that is, silica nanospheres (average diameter of about 150 nm) may be obtained.

Next, the prepared silica nanospheres (silica nanoparticles) may be washed with EtOH to remove excess reagents. Then, 50 μL of MPTS and 10 μL of ammonia hydroxide solution may be added to the silica nanospheres to functionalize the surface with thiol groups and as a result of it, it is possible to grow Ag nanoshells. The nanoparticles (silica nanoparticles) on which the Ag nanoshell is formed may be washed with EtOH to remove free thiol groups. Next, 30 mg of AgNO3 (silver nitrate) may be dissolved in 50 mL of ethylene glycol and then added dropwise to 60 μL of thiol-functionalized silica nanospheres (50 mg/mL). After adding the reducing agent hexadecylamine (0.603 g), bumpy AgNS (Ag nanoshell) may be obtained within 1 hour. After washing several times with EtOH to remove excess reagents, the bumpy AgNS may be functionalized with PDDA polymer as a final step. The bumpy AgNS may be dispersed in 30 mL of 0.05 v/v % PDDA aqueous solution, may be stirred for 1 hour, and then may be washed several times with EtOH. All of the above processes may be carried out at room temperature. The above-mentioned hexadecylamine may serve to enable activation of a 785 nm laser for allowing detection in the near-infrared region of the nanosensor to be realized.

The SERS nanosensor constructed as above may be a spherical nanoparticle with a SERS enhancement surface of approximately 300 nm in size, and its surface charge may be approximately +40 mV, and it may be suitable for detecting plant hormone substances by pulling small molecular substances of negatively charged plant hormones and placing them on the SERS enhancement surface through hydrogen bonding. Additionally, because it has high optical activity in the 785 nm region which may minimize the interference of strong fluorescence signals originating from plant chlorophyll, it may also be optimized for collecting optical signals within plants. The surface enhancement factor calculated through Raman signal measurement may reach approximately 107 times or more, and thus, trace amounts (nM level) of target substances (plant hormone substances) may be captured.

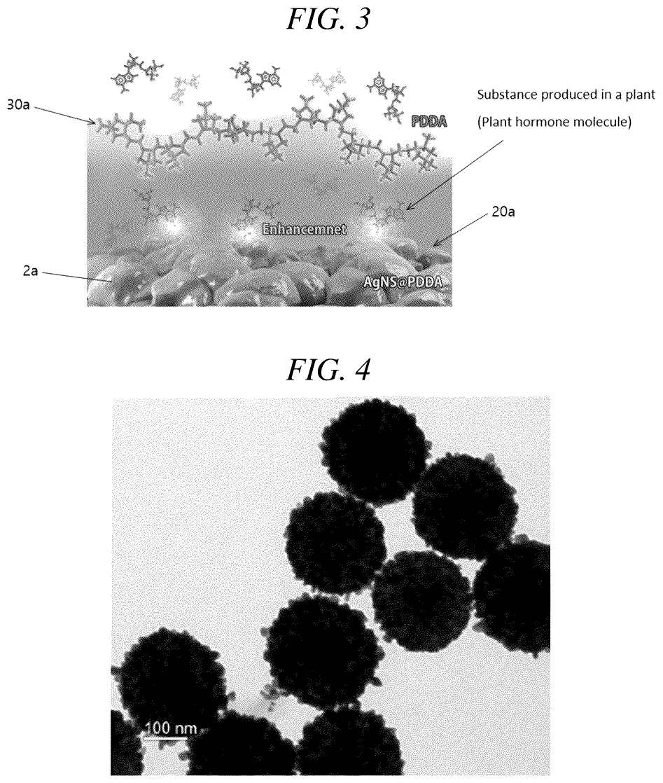

FIG. 3 is a conceptual diagram illustrating enhanced Raman scattering occurring on the surface of a SERS nanosensor according to an embodiment of the present invention. Referring to FIG. 3, an AgNS 20a made of alkylamine may greatly improve

Raman scattering and optimal SERS excitation in the near-infrared (NIR) region by creating a bumpy surface. A surface of AgNS 20a may be modified to increase water compatibility and bring several plant hormone molecules close to the nanosensor surface by introducing PDDA 30a which is a water-soluble cationic polymer.

FIG. 4 is a TEM (transmission electron microscope) image of a SERS nanosensor synthesized according to an embodiment of the present invention.

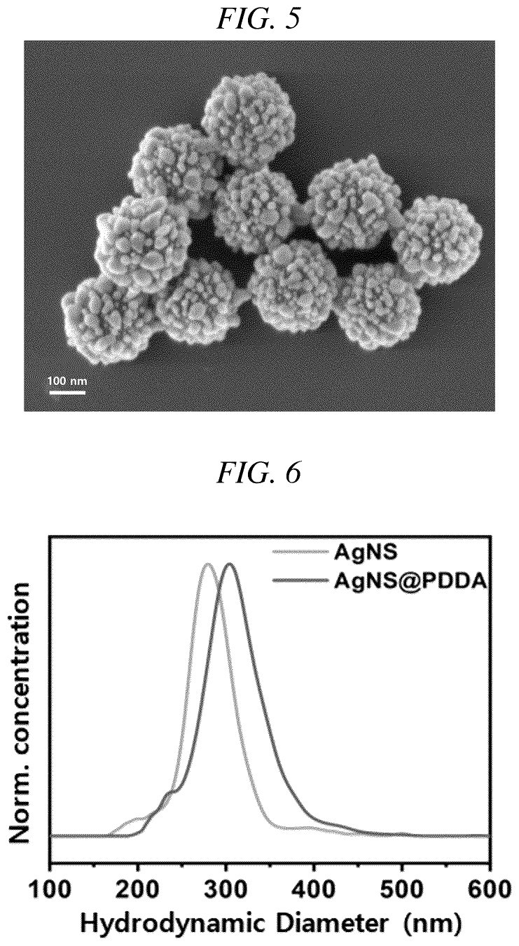

FIG. 5 is a scanning electron microscope (SEM) image of a SERS nanosensor synthesized according to an embodiment of the present invention.

Referring to FIG. 4 and FIG. 5, a SERS nanosensor containing AgNS functionalized with PDDA (i.e., AgNS@PDDA) has a bumpy surface and may have a diameter of about 300 nm.

FIG. 6 is a graph illustrating the results obtained by evaluating the hydrodynamic diameter of the SERS nanosensor synthesized according to an embodiment of the present invention and the structure according to the comparative example.

Referring to FIG. 6, it may be seen that the hydrodynamic diameter of the SERS nanosensor having a structure in which AgNS functionalized with PDDA (i.e., AgNS@PDDA) has increased by about 20 nm increased by about 20 nm as compared with a structure (i.e., AgNS) according to a comparative example in which only AgNS is formed on the surface of the nanoparticle without PDDA.

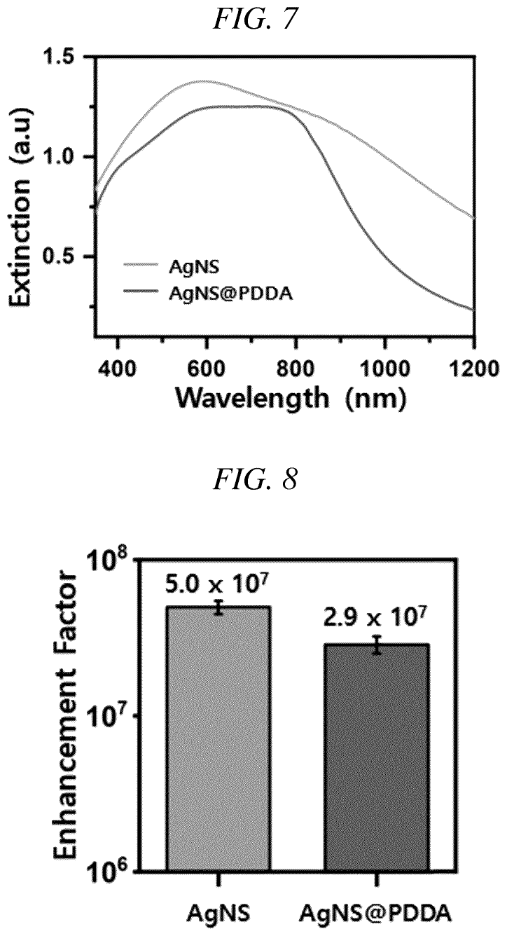

FIG. 7 is a graph illustrating comparison results of the UV-visible extinction spectrum of a SERS nanosensor synthesized according to an embodiment of the present invention and a structure according to a comparative example.

Referring to FIG. 7, it may be confirmed that in terms of UV-visible light extinction characteristics, the optical properties of the SERS nanosensor (i.e., AgNS@PDDA) synthesized according to the embodiment are similar to those of the structure (i.e., AgNS) according to the comparative example up to 800 nm. Therefore, in the case of the SERS nanosensor according to the embodiment, photoexcitation at 785 nm may be possible to collect the SERS spectrum of plant signaling molecules without interference from chlorophyll fluorescence.

FIG. 8 is a graph illustrating comparison results of the Raman enhancement factors of a SERS nanosensor (i.e., AgNS@PDDA) synthesized according to an embodiment of the present invention and a structure (i.e., AgNS) according to a comparative example.

Referring to FIG. 8, the estimated Raman enhancement factor of the SERS nanosensor (i.e., AgNS@PDDA) synthesized according to the embodiment may be sufficient to detect very small amount of plant hormone molecules, which is about 2.9×10⁷.

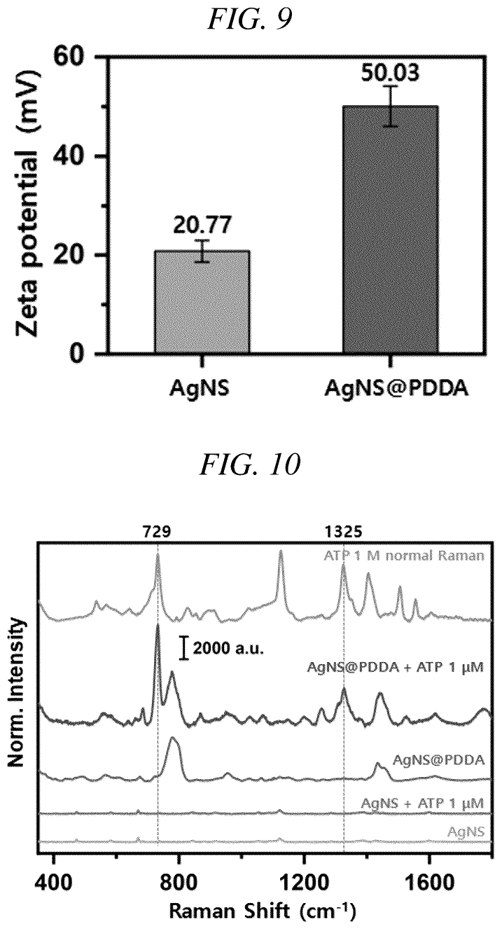

FIG. 9 is a graph illustrating comparison results of the zeta potential of a SERS nanosensor (i.e., AgNS@PDDA) synthesized according to an embodiment of the present invention and a structure (i.e., AgNS) according to a comparative example.

Referring to FIG. 9, the structure (i.e., AgNS) according to the comparative example had a positive zeta potential (+20 mV), and the SERS nanosensor (i.e., AgNS@PDDA) synthesized according to the embodiment had a higher surface charge (+50 mV) due to the 4^{th} ammonium moiety of PDDA. AgNS may have a positive charge due to the adsorption of alkyl amines used for Ag ion reduction. Some of these molecules were replaced with positively charged PDDA polymer, which is a polyelectrolyte for stabilizing noble metal nanoparticles. Replacing these small positive molecules with large positive molecules may be thermodynamically advantageous. The high surface charge of AgNS@PDDA may contribute to the excellent colloidal stability of the nanoparticles. When loosely wound PDDA polymer chains pull plant signaling molecules, the molecules may be positioned close to the AgNS surface, thereby producing a highly enhanced Raman signal.

FIG. 10 is a graph illustrating in vitro SERS spectra of AgNS, AgNS to which 1 μM ATP (adenosine triphosphate) was applied, AgNS@PDDA, and AgNS@PDDA to which 1 μM ATP was applied. FIG. 10 also includes the normal Raman spectrum of 1M ATP. Here, the AgNS refers to the structure according to the comparative example in which only AgNS is formed on the surface of the nanoparticle without PDDA, and the AgNS@PDDA refers to the SERS nanosensor according to the embodiment in which AgNS functionalized with PDDA is formed on the surface of the nanoparticle.

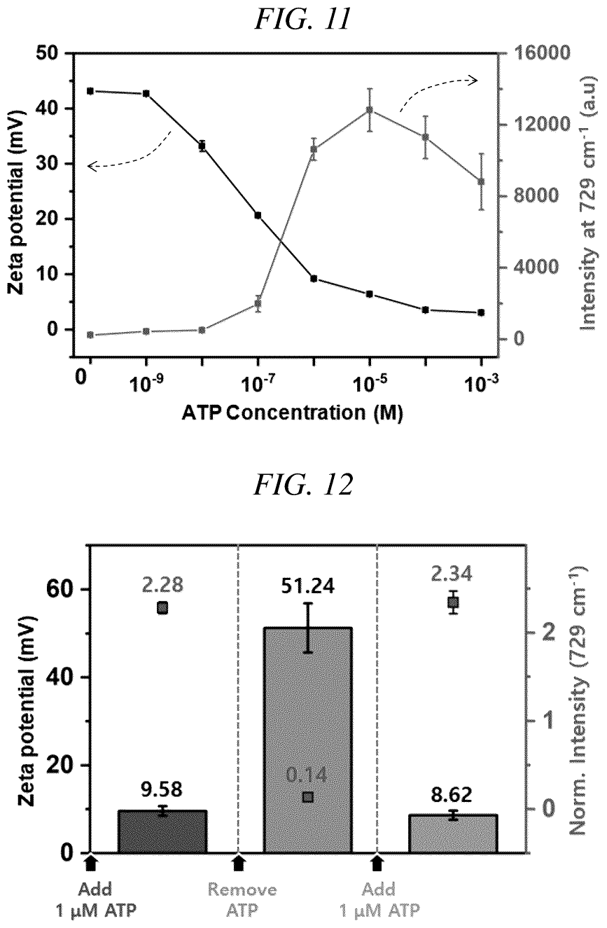

FIG. 11 is a graph illustrating changes in zeta potential (black color) and changes in SERS intensity (magenta) at 729 cm⁻¹ of AgNS@PDDA according to ATP concentration.

Referring to FIG. 10 and FIG. 11, extracellular adenosine-5-triphosphate (eATP) may be an essential target molecule for understanding plant stress and physiology. Because ATP has a small Raman cross-section and low affinity for a metal surface, it has not been easy to use Raman spectroscopy to monitor eATP in plant systems. Nevertheless, the AgNS@PDDA nanosensor according to an embodiment of the present invention may monitor the SERS signal of ATP by using limit of detection (LOD) of 10⁻⁸ M in aqueous conditions without the help of labeling molecules or aptamers.

PDDA molecules may attract ATP by electrostatic interactions and then trap ATP near the AgNS surface through the formation of multiple hydrogen bonds. Separate X-ray photoelectron spectroscopy (XPS) analysis showed that ATP molecules interacted with PDDA polymer chains.

ATP concentration-dependent changes in the zeta potential of AgNS@PDDA may also confirm the interaction between ATP molecules and the AgNS@PDDA surface. The positive charge of AgNS@PDDA continued to drop as the ATP concentration increased up to 10⁻⁶ M, and subsequently, the nanoparticle became neutral at ATP concentrations above 10⁻⁵ M, leading to nanoparticle agglomeration induced by reduced electrostatic repulsion. Nevertheless, the nanosensor may be stable in plant systems because the concentration of eATP is much lower than 10⁻⁶ M in plants.

FIG. 12 is a graph illustrating changes in zeta potential of the nanosensor according to the change in normalized SERS signal intensity of ATP upon addition or removal of 1 μM ATP.

Referring to FIG. 12, the response of the AgNS@PDDA sensor to the analyte ATP may be reversible. The surface charge of the nanosensor was found to be fully recovered by ATP removal and then dropped again upon addition of 1 μM ATP.

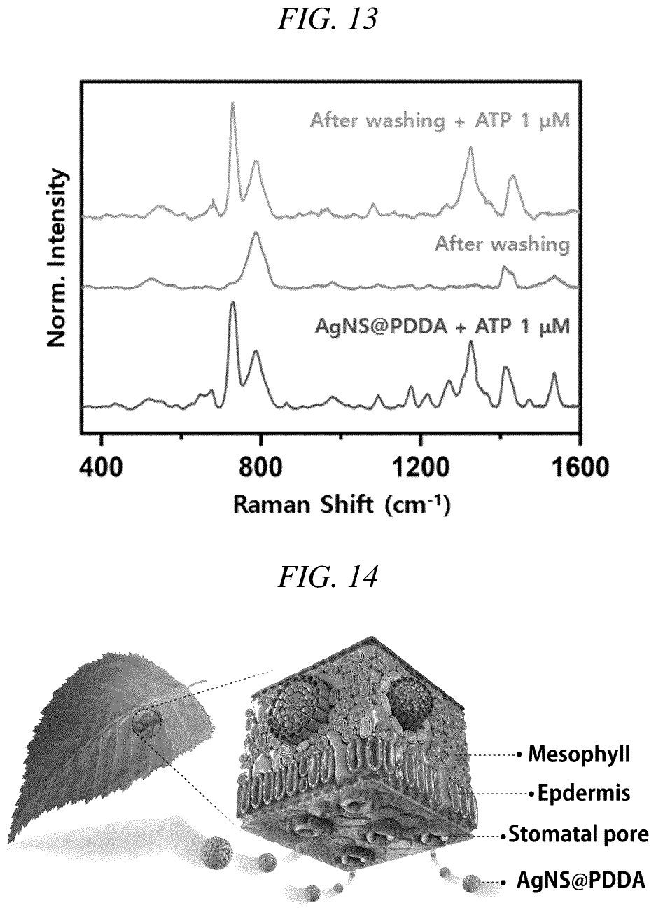

FIG. 13 is a graph illustrating changes in the SERS spectrum of the nanosensor depending on the presence or absence of 1 μM ATP. Here, the SERS spectrum was acquired with a 660 nm laser at 25 mV.

Referring to FIG. 13, the SERS spectrum disappeared when ATP was removed and

reappeared when ATP was added, which may correspond to a change in zeta potential. This reversible sensing mechanism could enable in vivo sensing applications which monitor the dynamics of endogenous phytochemicals, such as production, increase, and decrease in plants.

FIG. 14 is a schematic diagram illustrating infiltration of AgNS@PDDA nanosensors through stomatal pores of a plant and distribution of nanosensors in the cross section of plant leaves.

Referring to FIG. 14, the AgNS@PDDA nanosensor may be infiltrated into a leaf, which proves a fact that a nanosensor may be applied within a living plant.

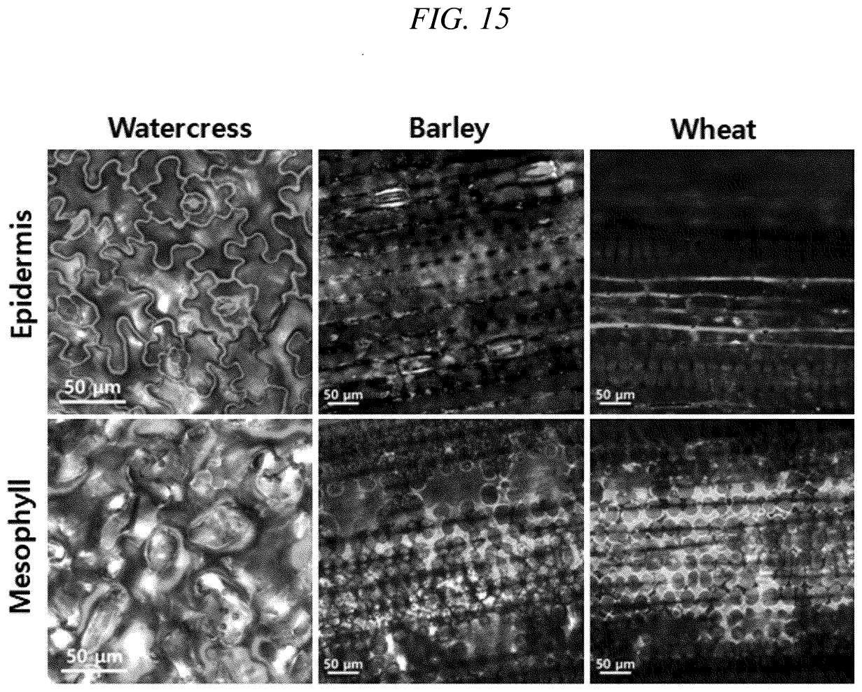

FIG. 15 shows CLSM (confocal laser scanning microscopy) images of watercress, barley, and wheat leaves into which dye-labeled silica nanoparticles (diameter 300 nm, 0.1 mg/mL) were introduced. The nanoparticles may localize with cell membranes, cell walls, and intercellular spaces.

Referring to FIG. 15, the positions of nanoparticles in plant leaves were confirmed by CLSM. When 300 nm-sized silica nanoparticles labeled with Alexa Fluor 488 dye were infiltrated into plant leaves, they were observed along with cell walls and membranes in the intercellular space of the epidermis and mesophyll of watercress, barley, and wheat. Dye-labeled nanoparticles are shown in a green color, and chloroplasts are shown in a red color.

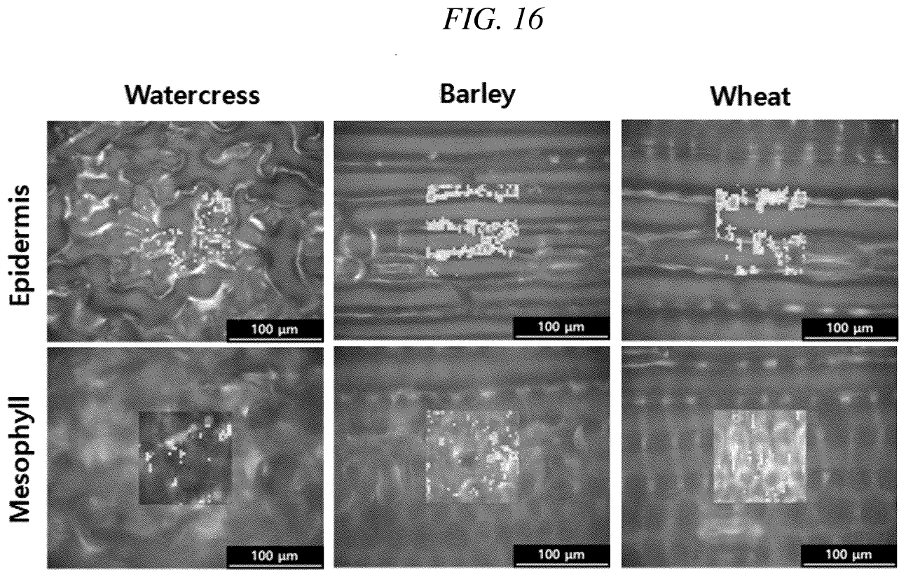

FIG. 16 is a diagram illustrating an overlay of brightfield images and confocal SERS intensity maps of the epidermis and mesophyll of watercress, barley, and wheat leaves. FIG. 16 shows measurements taken 2 hours after infiltrating the AgNS@PDDA (0.1 mg/mL) nanosensor into watercress, barley, and wheat leaves.

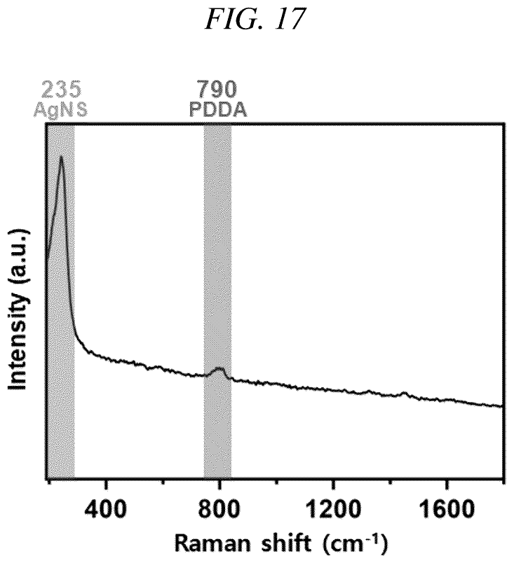

FIG. 17 is a graph illustrating the SERS spectrum of plant leaves infiltrated with AgNS@PDDA nanosensors.

Referring to FIG. 16 and FIG. 17, the SERS intensity map of the nanosensor-embedded leaf was obtained by using the strong SERS band of AgNS@PDDA at 235 cm⁻¹, which may correspond to Ag. . . . O stretch related to metal-solvent adsorption. It shows a strong band at 235 cm⁻¹ corresponding to Ag. . . . O stretch, and a relatively small band at 790 cm⁻¹ corresponding to PDDA.

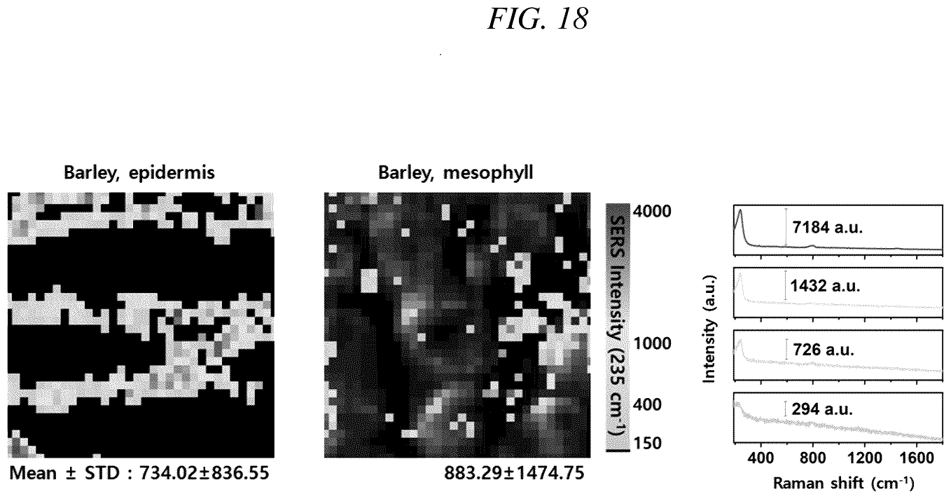

FIG. 18 is a diagram illustrating the confocal SERS intensity map of barley leaves infiltrated with AgNS@PDDA (0.1 mg/mL) and the spectra of selected spots having different SERS intensities. All SERS spectra were acquired with a 785 nm laser at 2 mW.

The SERS intensity map in FIG. 18 reconfirms that AgNS@PDDA nanosensor particles exist outside the plasma membrane in both of the epidermis and mesophyll layers.

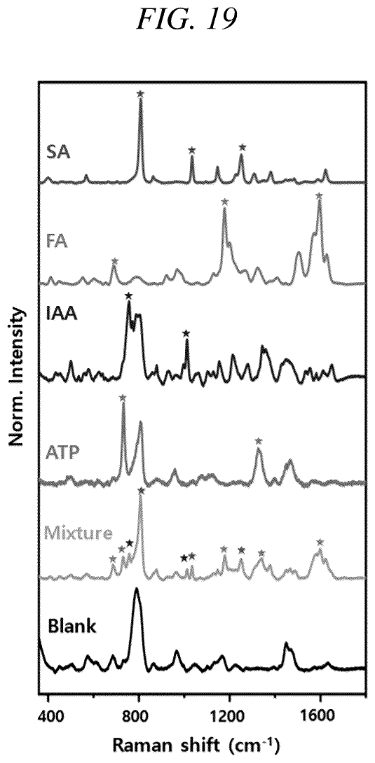

FIG. 19 is a graph illustrating SERS spectra of various plant hormone molecules. FIG. 19 includes SERS spectra of 10 μM SA, 10 μM FA, 100 μM IAA, 100 μM ATP, mixture, and AgNS@PDDA alone (Blank). The concentration of each molecule in the mixture was 2.5 μM SA, 2.5 μM FA, 25 μM IAA, and 25 μM ATP. Stars indicate characteristic bands contributed by SA (red), FA (cyan), IAA (blue), and ATP (green), respectively. Their unique SERS bands were distinguished in mixed solutions. The SESR spectrum is a representative result of five independent experiments. Here, SA represents salicylic acid, FA represents folic acid, IAA represents indole-3-acetic acid, and ATP represents adenosine triphosphate.

Referring to FIG. 19, because plants respond to stress stimuli in a multimodal manner, multiple detection and identification of target analytes may be essential for monitoring plant health or diagnosing plant diseases. SERS spectra of four representative plant signal molecules, ATP, SA, IAA, and FA were acquired in order to demonstrate the feasibility of the nanosensor for detecting multiple chemical analytes. IAA is one of the most common signal molecules in the auxin family which regulates plant growth and development. FA is essential for single-carbon transfer reactions and are known to mediate SA-dependent immunity in plants contributing to DNA synthesis in living organisms. A nanosensor may detect a variety of analytes and immediately identify them by their unique Raman fingerprints without labeling and using aptamers explicitly designed for each analyte. ATP has characteristic SERS bands at 729 corresponding to the adenosine ring and 1325 cm⁻¹, and SA shows a strong band at 808 cm⁻¹ and two moderately strong bands at 1035 and 1248 cm⁻¹. IAA has distinct bands at 755 and 1010 cm⁻¹, and FA has strong bands at 1178 and 1595 cm⁻¹ and a weak band at 690 cm⁻¹. The sensor platform may detect multiple analytes simultaneously by acquiring distinguishable SERS spectra unique to the hormone molecules in the mixture.

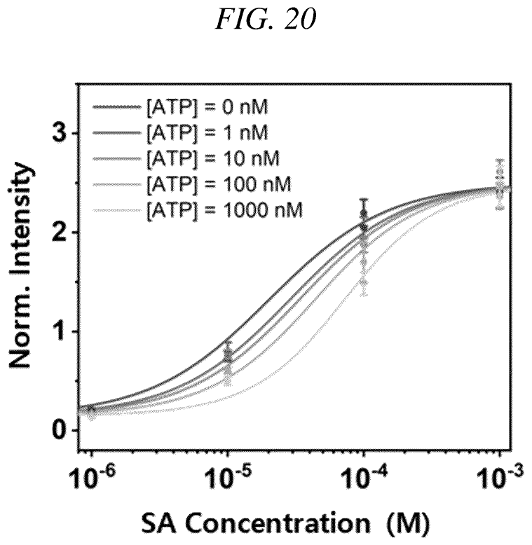

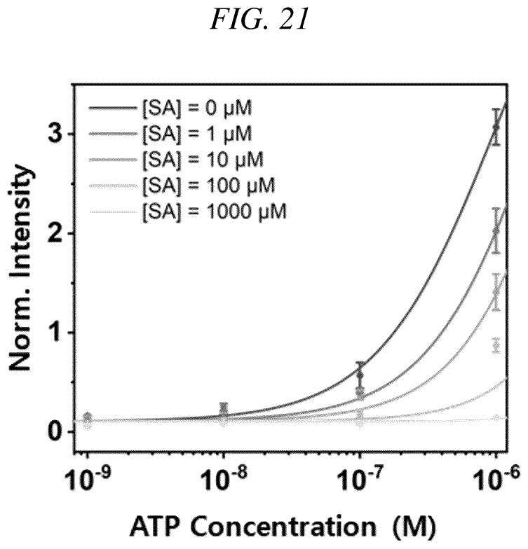

FIG. 20 and FIG. 21 are graphs illustrating concentration dependence of SERS intensity in binary mixtures. FIG. 20 shows intensity of the SERS band at 1035 cm⁻¹ as a function of SA concentration and the presence of other hormone molecules. FIG. 21 shows the intensity of the SERS band at 729 cm⁻¹ as a function of ATP concentration and the presence of other hormone molecules. The bands at 1035 and 729 cm⁻¹ represent SA and ATP, respectively.

Referring to FIG. 20 and FIG. 21, two sets of in vitro SERS measurements were performed by using AgNS@PDDA to acquire insights for the SERS signal of the target hormone molecule in the presence of other hormone molecules. One set included SERS measurements of SA at various concentrations at a constant ATP concentration, and the other set included SERS measurements with ATP at various concentrations with a constant SA concentration. As a result, the SERS intensity of SA at 1035 cm⁻¹ increased according to the concentrations and reached a plateau at 10⁻³ M SA, while the SERS intensity of ATP at 729 cm⁻¹ increased faster at very low ATP concentrations below 10⁻⁶ M.

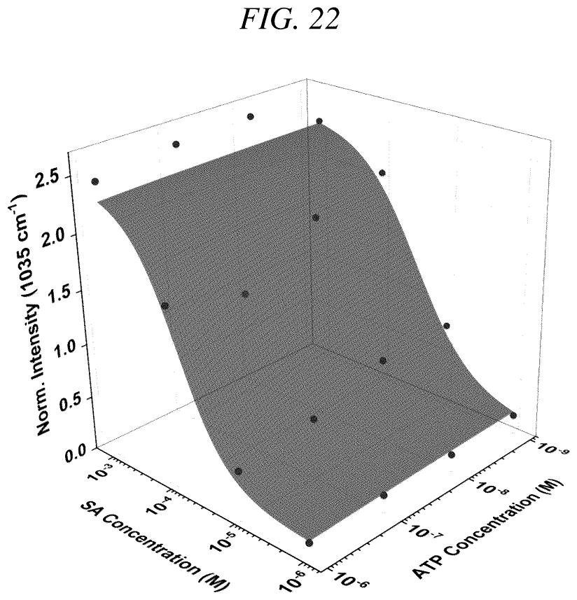

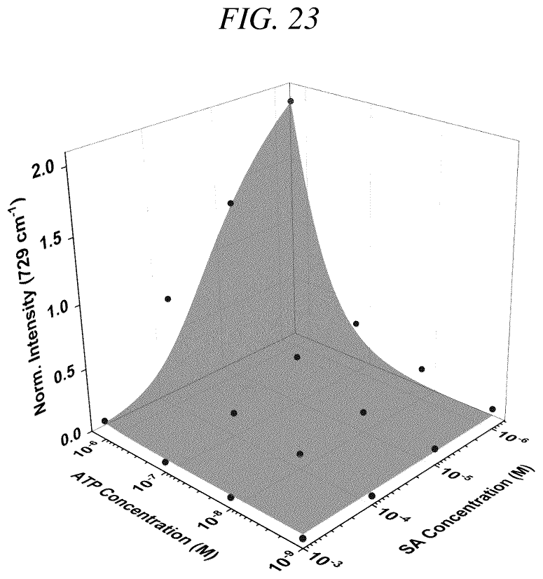

FIG. 22 and FIG. 23 are graphs illustrating 3D plots of the SERS bands of SA and TP according to their concentrations in a mixture of SA and ATP. The SERS band at 1035 cm⁻¹ corresponds to SA and the SERS band at 729 cm⁻¹ corresponds to ATP. All SERS spectra were acquired by using a 660 nm laser at 25 mW.

Referring to FIG. 22 and FIG. 23, the fitting results for the 3D surface plot using ATP and SA concentrations as independent variables show that the adjusted coefficient of determination (R2) for SA was obtained as 0.9888, and the adjusted coefficient of determination (R2) for ATP was obtained as 0.9781. This related model may be extended to apply to more complex environments such as plant fluids.

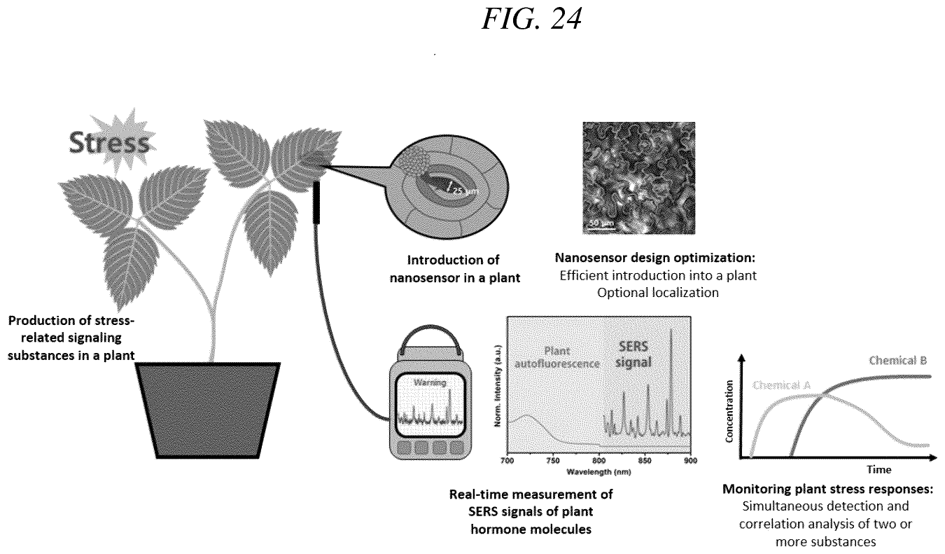

FIG. 24 is a diagram for explaining a plant monitoring apparatus using a SERS nanosensor according to an embodiment of the present invention and a plant monitoring method.

Referring to FIG. 24, the plant monitoring apparatus according to the embodiment may include a SERS nanosensor for detecting a substance produced within a plant and a Raman spectrometer for detecting a SERS signal generated from the SERS nanosensor. The plant monitoring method according to the embodiment may include a step for introducing the SERS nanosensor for detecting the substance produced in the plant into the living plant and a step for measuring the SERS signal generated from the SERS nanosensor by using Raman spectroscopy. For example, after introducing the SERS nanosensor into the leaves of a plant, a laser is irradiated to the leaves of the plant by using a Raman spectrometer and the SERS signal is detected from the reflected light so that the health status of the plant and the presence or absence of disease may be monitored. Plants may produce stress-related plant hormone molecules due to infection or injury, and the occurrence of specific plant hormone molecules may be detected early and in real time by using SERS nanosensors. At this time, two or more hormone substances may be detected simultaneously, and their correlation may also be analyzed.

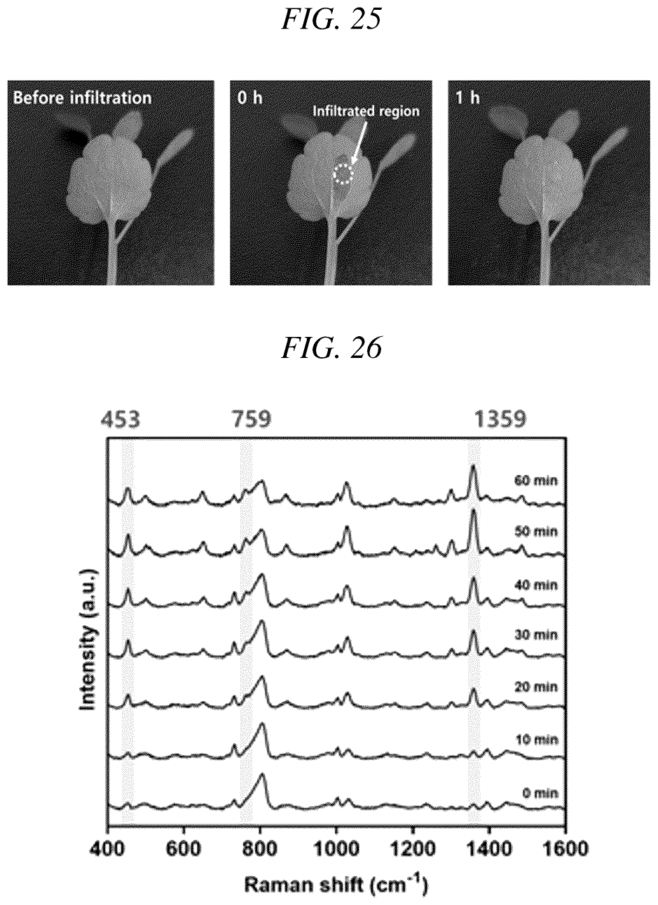

FIG. 25 is a photographic image illustrating a leaf of watercress into which a SERS nanosensor was introduced according to an embodiment of the present invention.

FIG. 26 is a graph illustrating the results obtained by detecting the SERS signal generated due to the substances (molecules) generated by wound stimulation for watercress into which a SERS nanosensor was introduced.

If the SERS nanosensor according to an embodiment of the present invention is used, for example, SA (salicylic acid), a major plant hormone produced by biotic stress, and eATP (extracellular adenosine triphosphate) produced by abiotic/biotic stress in wheat and barley infected with fungal diseases may be detected simultaneously. When several hours passed after infection with a fungal disease (on the day of inoculation), it is difficult to distinguish the difference from uninfected plants with the naked eye, but in plants introduced with SERS nanosensors (optical nanosensors), early diagnosis is made by detecting SA-related signals after infection. Additionally, it may be possible to detect signals from the fungus itself on the second or third day of infection.

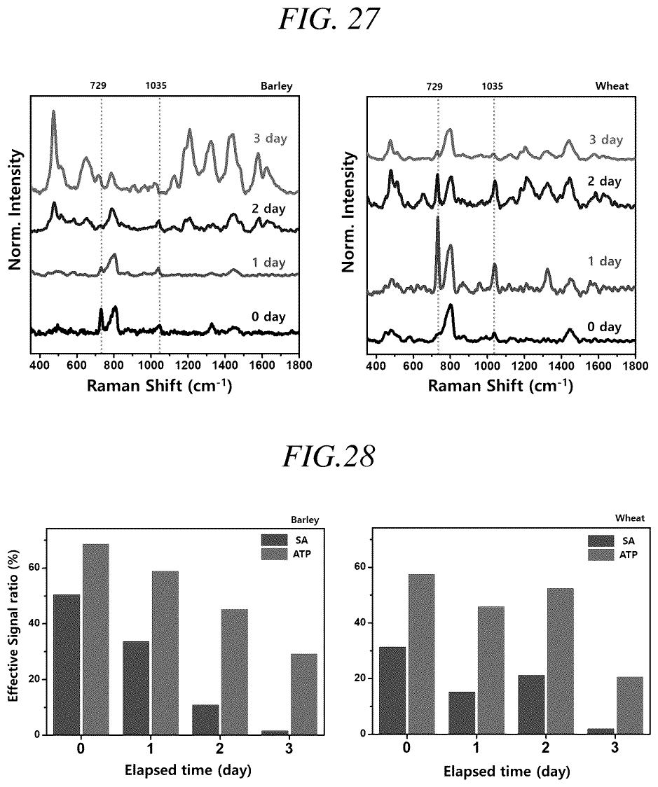

FIG. 27 is a graph illustrating the results obtained by measuring changes in Raman signals after fungal infection in wheat and barley according to an embodiment of the present invention.

Referring to FIG. 27, SA (808 cm⁻¹) and eATP (729 cm⁻¹, 1035 cm⁻¹) signals were detected several hours after inoculation (0 day), and on the 3rd day, fungal signals (1200˜1600 cm⁻¹) was detected to a very large extent.

FIG. 28 is a graph illustrating the results obtained by measuring the detection frequency of plant hormones obtained from wheat and barley over time after fungal infection in wheat and barley according to an embodiment of the present invention.

Referring to FIG. 28, it may be seen that plant hormones are most often detected in the area around inoculation on the day of infection.

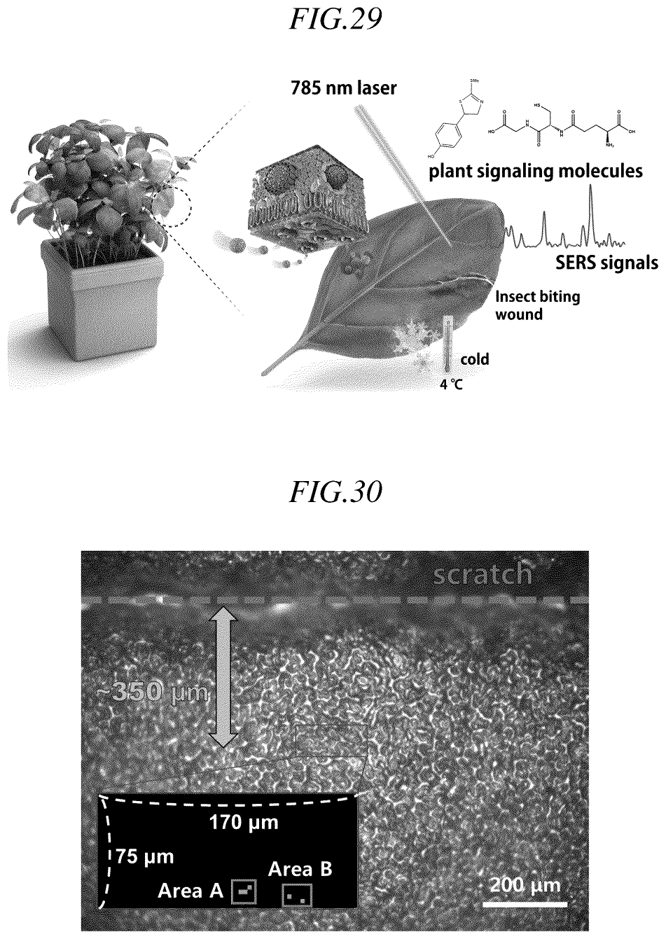

FIG. 29 is a diagram for explaining detection of SERS signals in living plants subjected to abiotic stress such as cold or wound.

Referring to FIG. 29, after introducing the SERS nanosensor according to the embodiment into a living plant, it may be possible to detect SERS signals for the substances (molecules) generated by stress such as cold or wounds.

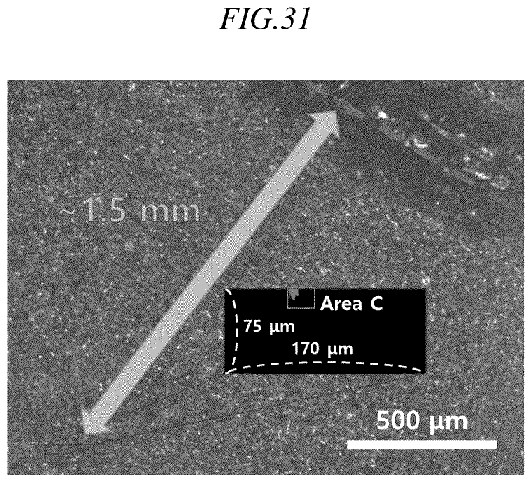

FIG. 30 and FIG. 31 are diagrams illustrating brightfield images of a small area of a leaf where SERS area scanning was performed. The inserts in FIG. 30 and FIG. 31 are SERS intensity maps obtained from SERS acquisition.

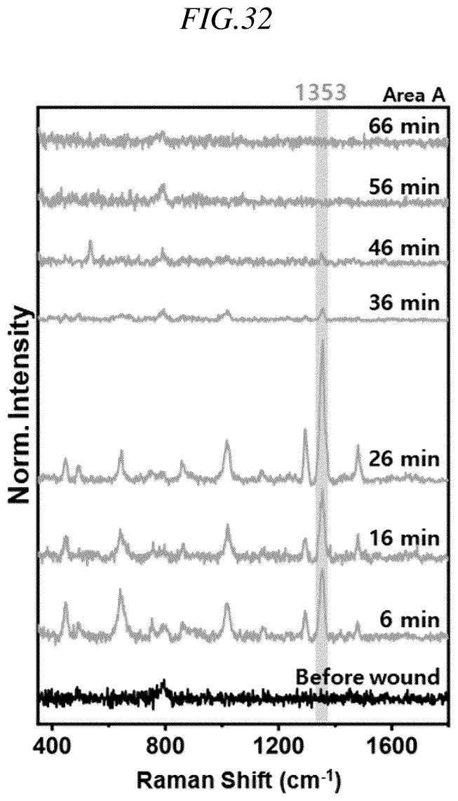

FIG. 32 to FIG. 34 are graphs illustrating Raman spectra obtained in region A, region B, and region C, respectively, at specific time intervals after wounding the leaf. Here, the areas A and B may correspond to areas A and B shown in FIG. 30, and the area C may correspond to area C shown in FIG. 31.

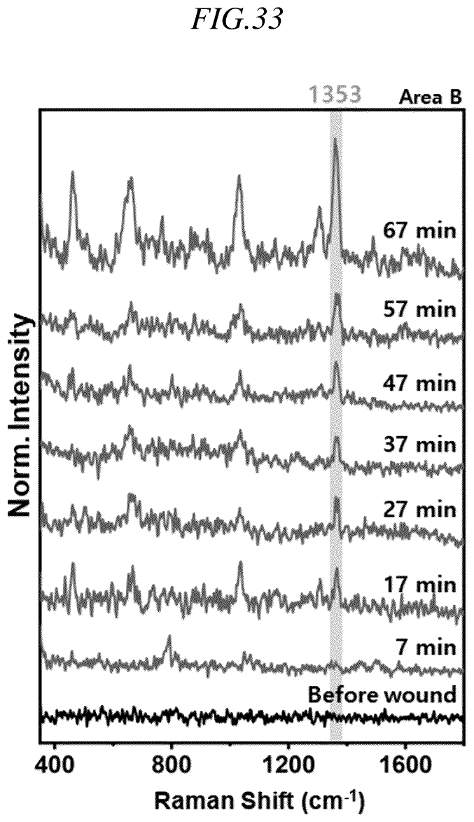

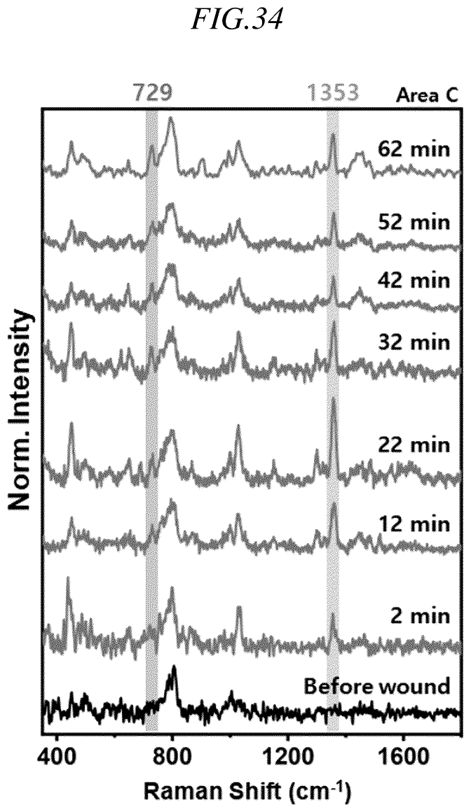

Referring to FIG. 30 to FIG. 34, it may be seen that the SERS signal at the point marked as A rapidly increased and lasted for 26 minutes and then disappeared for the next 10 minutes (FIG. 32). On the other hand, the SERS signal in B and C gradually increased and lasted for more than 1 hour (FIG. 33 and FIG. 34). The nanosensor at C may detect signaling molecules earlier as compared with the points A or B, which are closer to the wound site. Fluctuations in signal intensity may indicate that signal molecules have been produced and transmitted.

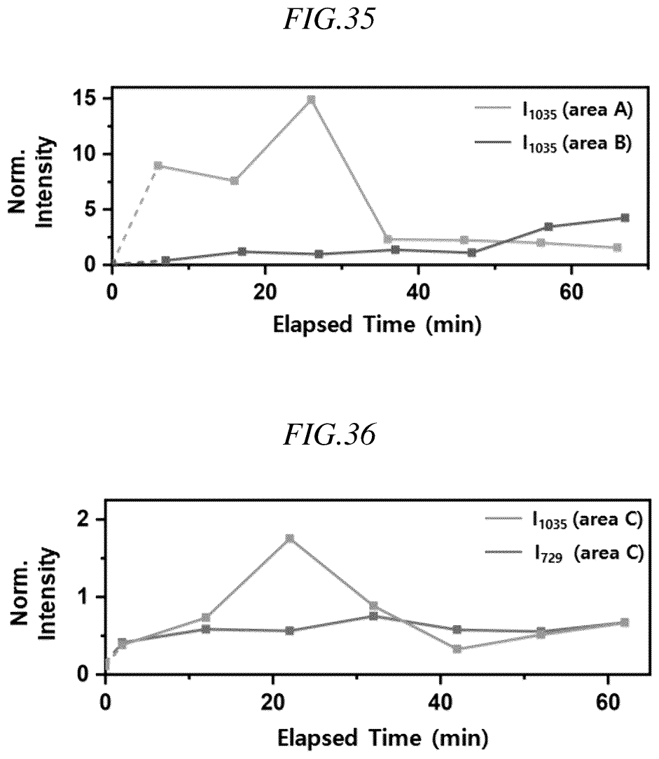

FIG. 35 and FIG. 36 are graphs illustrating a temporal profile of the SERS band at 1353 cm⁻¹ or 729 cm⁻¹ related to cruciferous phytoalexin or eATP obtained from the three regions A, B and C described above. FIG. 35 shows the results for areas A and B, and FIG. 36 shows the results for area C.

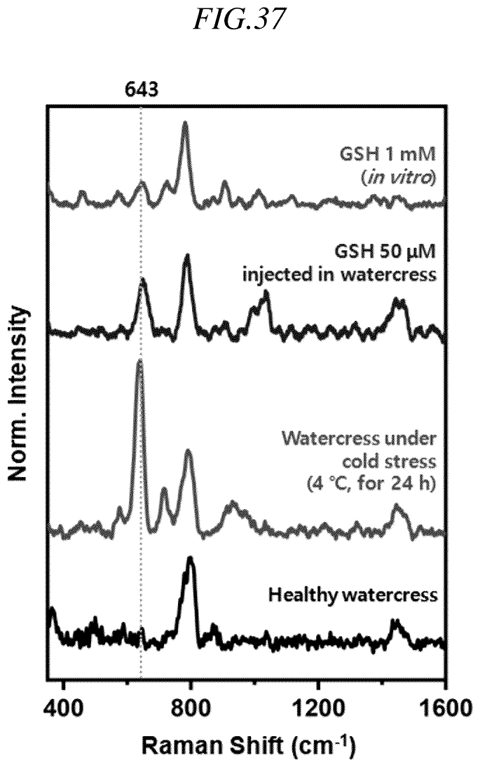

FIG. 37 is a graph illustrating the SERS spectrum obtained from in vitro 1 mM GSH (green), a leaf (blue) infiltrated with 50 μM GSH, a leaf (red) of plants after chilling stress for 24 hours at 4° C., and a control plant leaf (grown under normal conditions, black). Here, GSH represents glutathione.

Referring to FIG. 37, the nanosensor according to the embodiment may monitor endogenous chemicals produced by plants subjected to low temperature stress. Glutathione is a good indicator for understanding plant responses for chilling and cold acclimation, but measurements may be difficult to obtain due to the rapid oxidation of glutathione during sample preparation or the low sensitivity of detection tools. The nanosensor according to the embodiment has excellent advantages due to the strong interaction between the Ag nanoshell surface and the sulfhydryl group of glutathione. As a result, the nanosensors may easily probe endogenous glutathione molecules in living plants. The SERS signal appeared at 643 cm⁻¹, which corresponds to the glutathione produced when live watercress plants containing AgNS@PDDA were exposed to low temperature during storage at 4° C. for 24 hours. The nanosensor is located next to the cell wall and may preferentially monitor glutathione molecules moving across the cell wall.

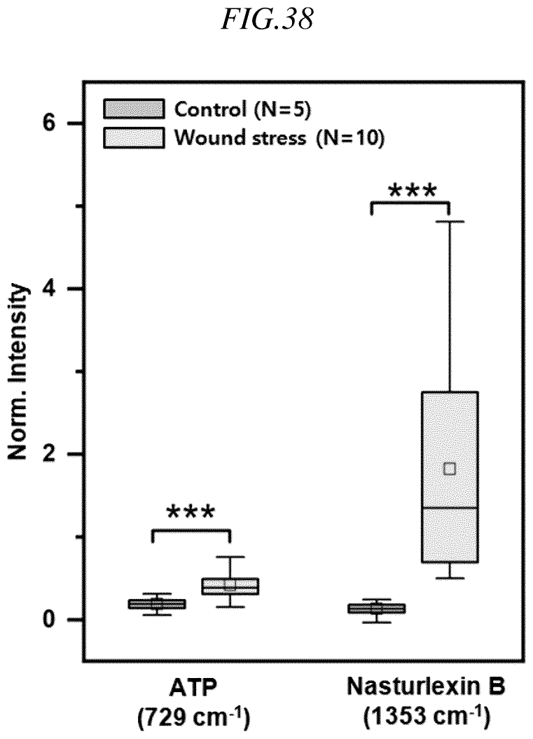

FIG. 38 is a graph illustrating signal comparison between control watercress plants and plants under wounding conditions. Here, control refers to uninjured plants. The intensities of the bands assigned to ATP at cm⁻¹ and nasturlexin B at cm⁻¹ were normalized to the intensity of 790 cm⁻¹ corresponding to the PDDA band of the nanosensor. Asterisks indicate values which are remarkably different from the control.

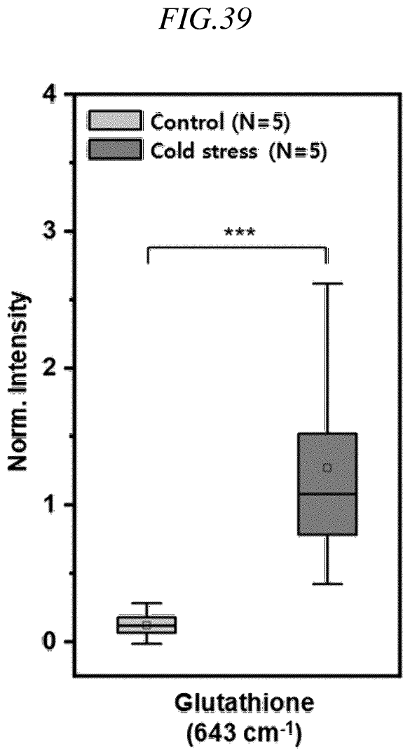

FIG. 39 is a graph illustrating signal comparison between control watercress plants and plants under cold stress conditions. Here, control refers to plants that were not subjected to chilling stress. The intensity of the band assigned to glutathione at 643 cm⁻¹ was normalized to the intensity at 790 cm⁻¹, corresponding to the PDDA band of the nanosensor. Asterisks indicate values which are remarkably different from the control.

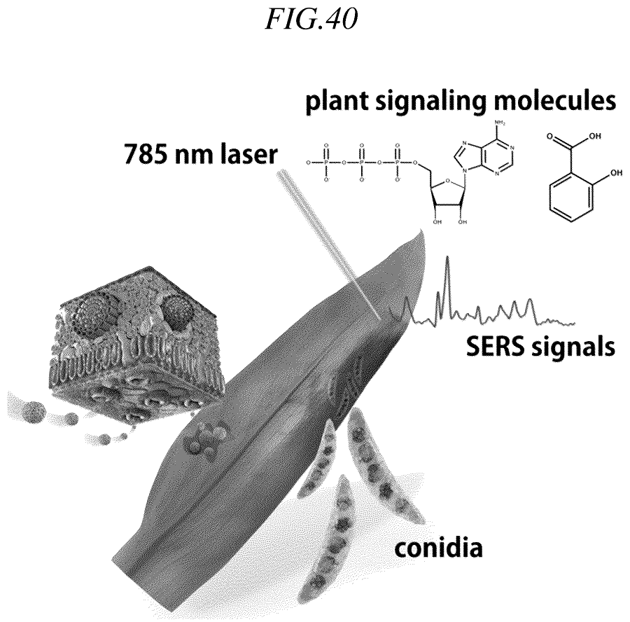

Referring to FIG. 38 and FIG. 39, as a result of comparing the normalized intensities of signaling molecules, it was found that the SERS signals of plants under wound or cold stress conditions were enormously different from the signals of healthy plants. FIG. 40 is a diagram schematically illustrating a SERS-based monitoring method for signaling molecules in living crops infected with fungi.

Referring to FIG. 40, after introducing the SERS nanosensor according to the embodiment into a living plant, it may be possible to detect SERS signals for substances (molecules) generated due to fungal infection.

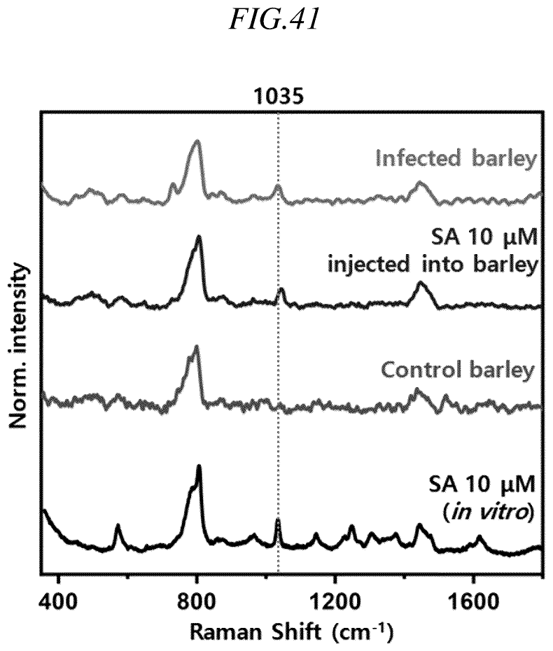

FIG. 41 is a graph illustrating the SERS spectrum obtained from barley leaves to confirm the 1035 cm⁻¹ band due to SA. FIG. 41 contains spectra obtained in vitro from 10 μM SA (black), untreated barley leaves (red), barley leaves infiltrated with 10 μM SA (blue), and barley leaves infected with F. graminearum (magenta).

Referring to FIG. 41, SA was infiltrated into barley leaves at various concentrations and SERS spectra of the SA-infiltrated leaves were collected in order to confirm whether the nanosensor may detect SA in plants. When SA was infiltrated into the leaves at a concentration of 10 μM or higher, a clear SERS peak appeared at 1035 cm⁻¹ which is characteristic of SA.

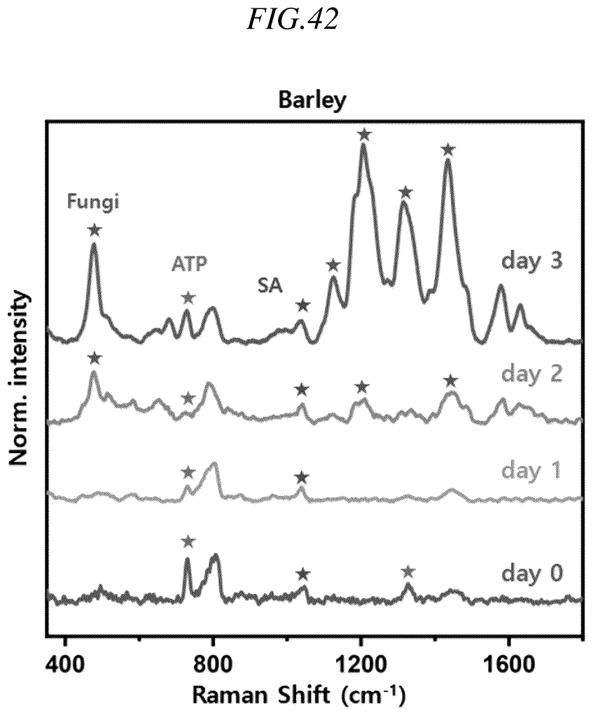

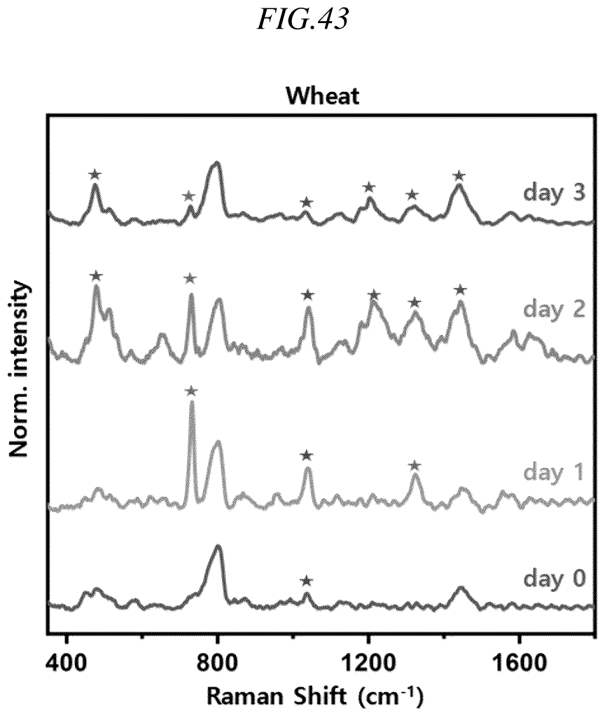

FIG. 42 and FIG. 43 are graphs illustrating representative SERS spectra obtained while monitoring the progression of fungal disease in infected barley and wheat plants, respectively.

Referring to FIG. 42 and FIG. 43, the nanosensor infiltrated into the leaf at a position 1 to 2 cm lower than a position where F. graminearum was inoculated. The nanosensor detected both of SA and eATP signals at 1035 cm⁻¹ (SA) and 729 cm⁻¹ (ATP) in barley and wheat leaves, respectively, 2 hours after F. graminearum inoculation (day 0). From day 0 (2 hours after inoculation), the nanosensor (AgNS@PDDA) detected SA and eATP signals for early diagnosis. From the 2nd day, the pathogen F. graminearum along with ATP and SA could be detected by using the nanosensor (AgNS@PDDA).

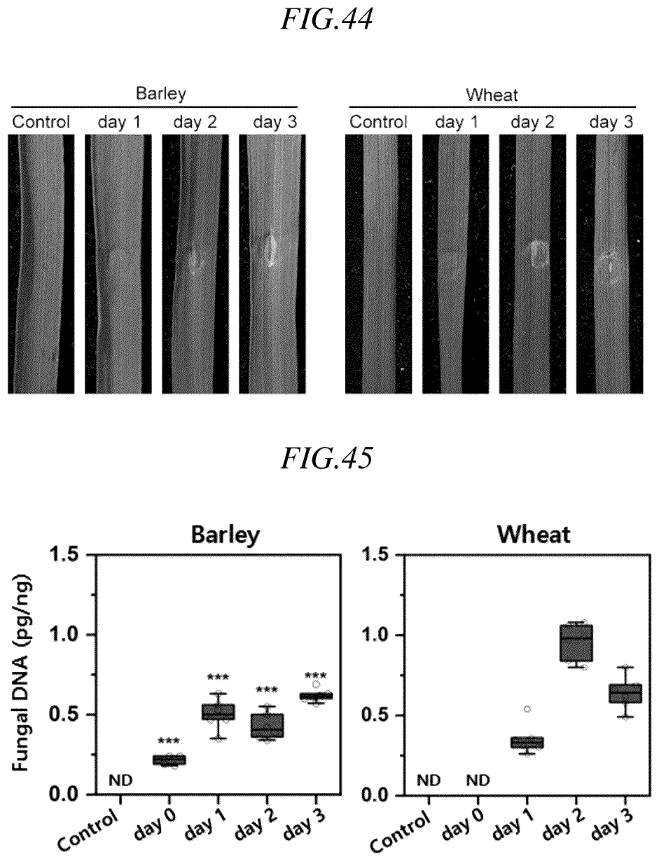

FIG. 44 is a photographic image illustrating lesion formation caused by fungal pathogens on barley and wheat leaves.

Referring to FIG. 44, barley and wheat leaves were inoculated with the fungal pathogen F. graminearum. Untreated plants are controls. Pictures were taken on day 0 (2 hours after vaccination), 1, 2, and 3. It may be seen that lesions began to form on the barley and wheat leaves from the 2nd day.

FIG. 45 is a graph illustrating the real-time PCR (polymerase chain reaction) analysis results of fungal DNA content during disease progression. The amount of F. graminearum DNA was expressed as picograms per nanogram of plant DNA. ND means a state of “no detection”.

Referring to FIG. 45, Real-Time PCR was able to detect fungal DNA from day 0 (2 hours after inoculation) in barley, but it could be detected on day 1 in wheat. The result of Real-time PCR analysis which is a standard pathogen detection method showed that although the amount of fungal DNA increases over time, the mass of fungal DNA assessed in plant tissue was less than 0.1% of plant DNA. After 30 cycles of PCR, fungal DNA was only slightly detected in barley (limit of detection, 0.2 pg ng⁻¹), whereas it was not detected on day 0 and was close to the limit of detection on day 1 in wheat.

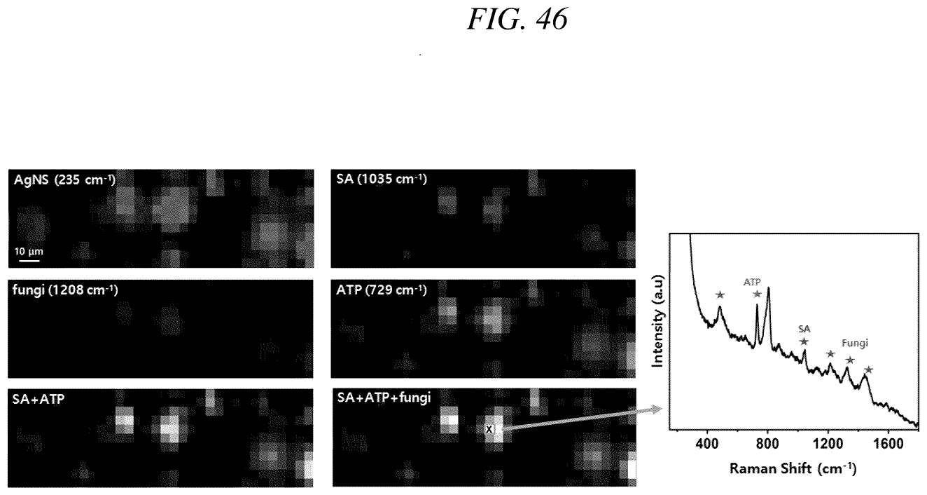

FIG. 46 shows a SERS intensity map obtained from infected wheat plants on day 2. Here, the SERS signal was displayed as a color map. Purple corresponds to AgNS (235 cm⁻¹), red to SA (1035 cm⁻¹), blue to mold (1208 cm⁻¹), and green to ATP (729 cm⁻¹). The raw SERS spectrum (no-baseline correction) for multiple molecular signals at the cross-marked points of the SERS intensity map is graphed on the right. All SERS spectra were acquired by using a 785 nm laser at 2 mW.

Referring to FIG. 46, the SERS intensity map of a leaf embedded with nanosensors visualizes simultaneous and multiple detection of various defense signal molecules in living plants, enabling real-time spatial monitoring of plant defense signal responses to pathogens. SERS intensity maps were obtained on the second day of infection by using the SERS bands of SA and eATP together with the nanosensor AgNS@PDDA according to the embodiment, and were displayed as false-colored images. Although CATP and SA are detected simultaneously at the same location, not all locations may exhibit pathogen signals. This could mean that even plant cells which are not directly affected by fungal infection produce signaling molecules in response to the pathogen.

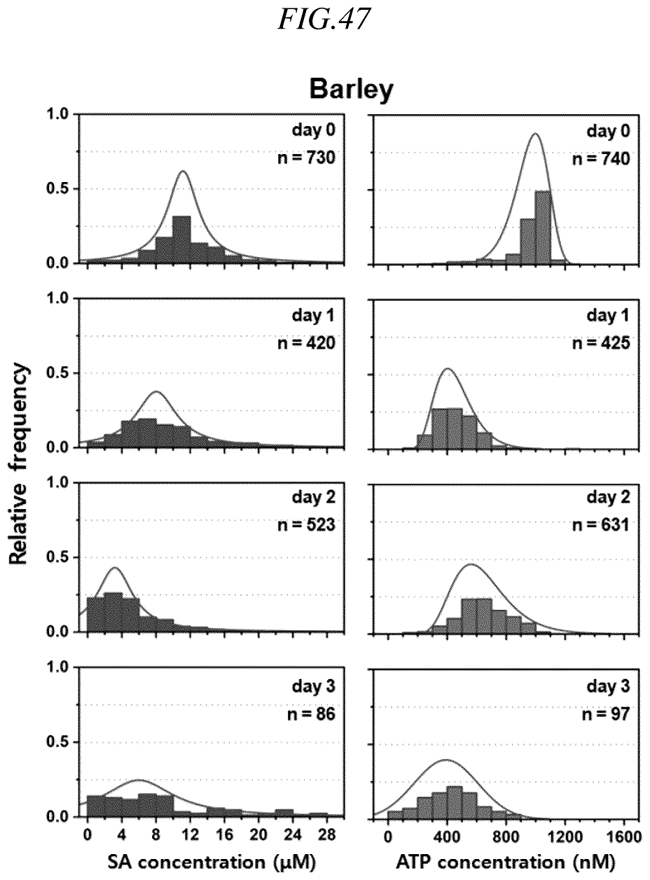

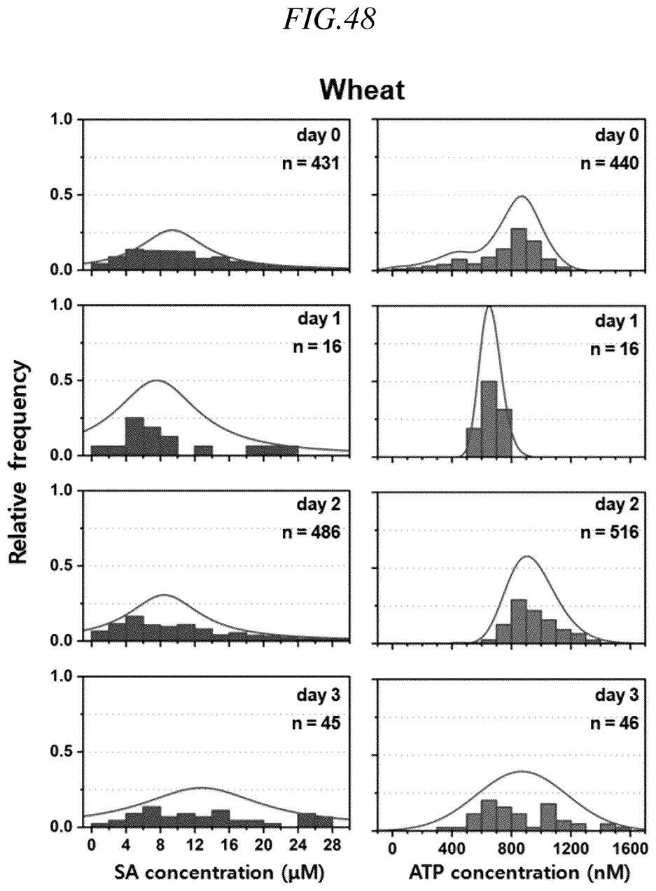

FIG. 47 and FIG. 48 are graphs illustrating representative histograms for presumptive concentrations of SA (red) and ATP (green) in living barley and wheat plants infected with F. graminearum, respectively. Each data point was calculated from a pixel of the SERS mapping image by using the calibrated 3D surface described in the text. In the histogram, n represents the number of data points of SERS mapping images obtained from four biologically independent plants.

Referring to FIG. 47, after inoculation of F. graminearum into barley and wheat plants, a change trend of SA concentration and eATP concentration over time may be confirmed.

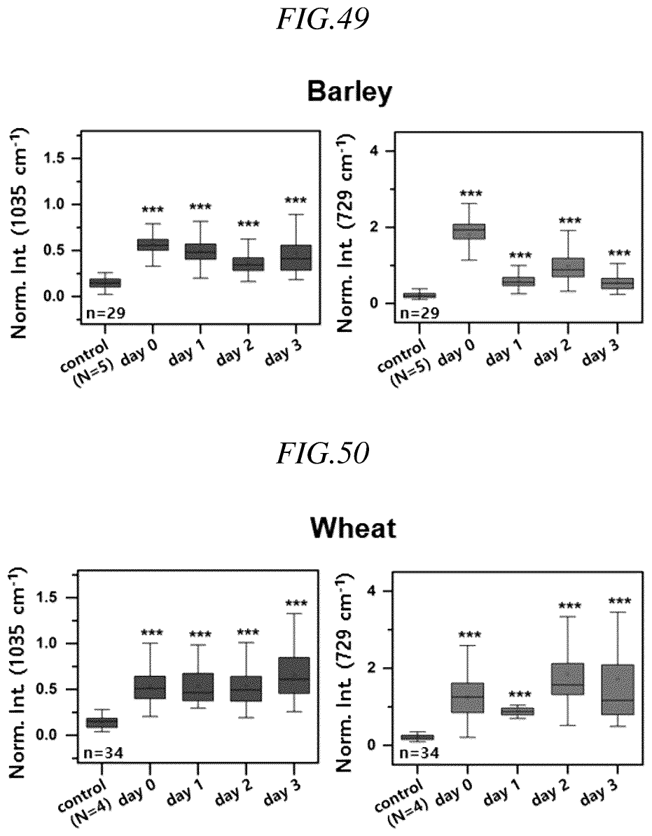

FIG. 49 and FIG. 50 are graphs obtained by comparing the signals between control crop plants and plants under fungal infection condition. Here, control refers to uninfected plants. The intensities of the bands assigned to ATP at 729 cm⁻¹ and SA at 1035 cm⁻¹ were normalized to the intensity of 790 cm⁻¹ corresponding to the PDDA band of the nanosensor. Asterisks indicate values that are remarkably different from the control. All SERS spectra were acquired by using a 785 nm laser at 2 mW.

Referring to FIG. 49 and FIG. 50, it may be seen that there is a tremendous difference between the signal in the uninfected plant (control) and the signal in the plant under fungal infection conditions. Considering that SERS signals for SA or ATP have never been observed in healthy barley and wheat plants when using nanosensors, the SERS spectral features of the nanosensors after fungal inoculation suggest that pathogen infection produces SA and eATP in infected tissues, thereby cause SAR (systemic acquired resistance).

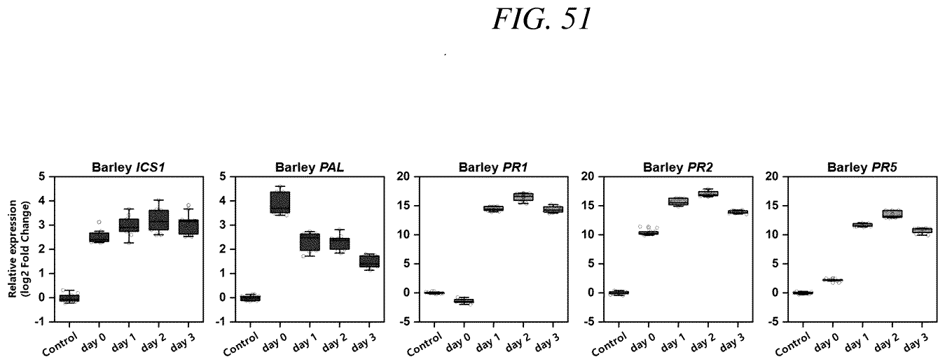

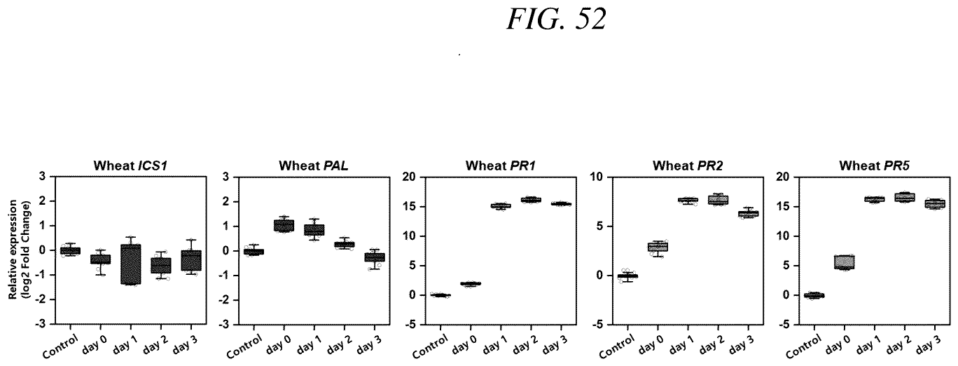

FIG. 51 and FIG. 52 are graphs illustrating the expression of ICS1 (isochorismate synthase), PAL (phenylalanine ammonia lyase), and selected PR (pathogenesis-related) genes induced in barley and wheat leaves inoculated with F. graminearum. Here, control refers to uninfected plants (control group). Log2-fold change values were generated by comparing gene expression with that of the control group at each infection time point. All measurements were normalized to the expression of the Actin gene. Asterisks indicate values that are remarkably different from the control.

Referring to FIG. 51 and FIG. 52, a real-time quantitative reverse transcription PCR (RT-qPCR) was performed on plant tissues inoculated with F. graminearum was performed in order to determine whether fungal infection induces SA biosynthesis and plant defense genes. One of two biosynthetic branches may produce SA, one contains isochorismate synthase (ICS) and the other contains phenylalanine ammonia lyase (PAL). It is found that the SA synthesis genes ICS1 and PR1 were rapidly induced, and their expression levels increased 2 hours after inoculation. This rapid SA synthesis gene expression is consistent with the results obtained by use of a nanosensor which detected SA signals only a few hours after F. graminearum inoculation.

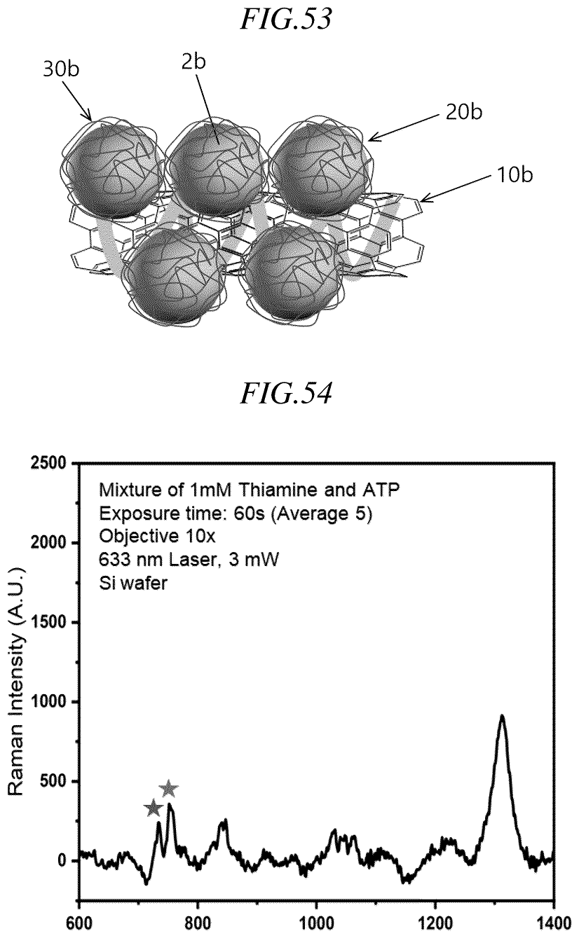

FIG. 53 is a diagram illustrating a SERS nanosensor for detecting a substance produced in a plant according to another embodiment of the present invention.

Referring to FIG. 53, the SERS nanosensor of this embodiment may include a first nanostructure 10b, a second nanostructure 20b containing a metal, which is disposed on a surface of the first nanostructure 10b and causes (induces) SERS, and a polymer material 30b which is bonded to a surface of the second nanostructure 20b and generates an attraction force that attracts the substance produced in the plant.

The first nanostructure 10b may have a nanotube shape. For example, the first nanostructure 10b may be or include a CNT (carbon nanotube). The CNT may include, for example, a single-walled carbon nanotube (SWNT), but is not limited thereto. The length of the first nanostructure 10b may be from several tens of nm to several um, and the outer diameter (diameter) may be from several nm to several hundreds of nm. As a specific example, the length of the first nanostructure 10b may be about 20 nm to 1 μm, and the outer diameter (diameter) may be about 2 nm to 100 nm.

The second nanostructure may include a plurality of nanoparticles 2b. The nanoparticle 2b may be an element for causing (inducing) SERS for the substance produced within the plant. The nanoparticle 2b may include or be composed of metals such as Au and Ag. For example, the nanoparticle 2b may be an Au nanoparticle. The nanoparticle 2b may have a length smaller than that of the first nanostructure 10b. The diameter of the nanoparticle 2b may be hundreds of nm or less or tens of nm or less. For example, the diameter of the nanoparticle 2b may be about 1 nm or more and about 300 nm or less. However, the diameter range of the nanoparticle 2b is exemplary and may vary depending on the case. A plurality of nanoparticles 2b may be assembled on the surface of the first nanostructure 10b. The second nanostructure 20b may serve to amplify SERS enhancement.

According to one example, the first nanostructure 10b may include a CNT and the second nanostructure 20b may include a plurality of Au nanoparticles 2b disposed on the surface of the CNT. The first nanostructure 10b may serve as a base for forming the second nanostructure 20b on its surface. Additionally, the first nanostructure 10b may be bonded to the second nanostructure 20b to enhance the plasmon effect.

The polymer material 30b may be formed on the surface of the second nanostructure 20b and may serve to generate an attraction force that attracts the substance produced within the plant. It may be said that the surface of the second nanostructure 20b is functionalized or modified by the polymer material 30b. The polymer material 30b may have a predetermined positive (+) charge. The polymer material 30b may be, for example, PDDA [poly(diallyldimethylammonium chloride)] or may include it. When PDDA is applied as the polymer material 30b, PDDA may effectively perform the role to attract (pull) the substances produced within the plant, and as a result, the detection characteristics of substances produced within the plant using SERS may be greatly improved. However, the polymer material 30b is not limited to PDDA and may change depending on the case.

According to one example, the SERS nanosensor according to this embodiment may have a nanoprobe structure in which Au nanoparticles (PDDA@AuNP) surface-modified with PDDA polymer are assembled on the surface of single-walled carbon nanotubes (SWNT), a “PDDA@AuNP-SWNT” structure. These SERS nanosensors may have a Raman enhancement factor of about 2.19×10⁶.

A manufacturing method of a SERS nanosensor for detecting a substance produced in a plant according to an embodiment of the present invention may include a step for preparing a first nanostructure, a step for forming a second nanosensor which is disposed on the surface of the first nanostructure, contains a metal and causes SERS, and a step for binding a polymer material which generates an attraction force that attracts the substance produced in the plant to the surface of the second nanostructure. For example, the first nanostructure may include a carbon nanotube (CNT), and the second nanostructure may include a plurality of Au nanoparticles disposed on the surface of the CNT. The polymer material may include PDDA [poly(diallyldimethylammonium chloride)]. First of all, after disposing the second nanostructure (nanoparticle) on the surface of the first nanostructure, the polymer material may be bound to the surface of the second nanostructure (nanoparticle). Alternatively, first of all, after the polymer material may be bound to the surface of the second nanostructure (nanoparticle), the second nanostructure (nanoparticle) to which the polymer material is bound may be formed on the surface of the first nanostructure.

FIG. 54 is a graph of a SERS spectrum illustrating the characteristics of simultaneously detecting ATP and thiamine by using the SERS nanosensor according to the example of FIG. 53.

Referring to FIG. 54, two star marks indicate peaks corresponding to ATP and thiamine. ATP and thiamine may be produced in plants under stress conditions, and they may be detected simultaneously.

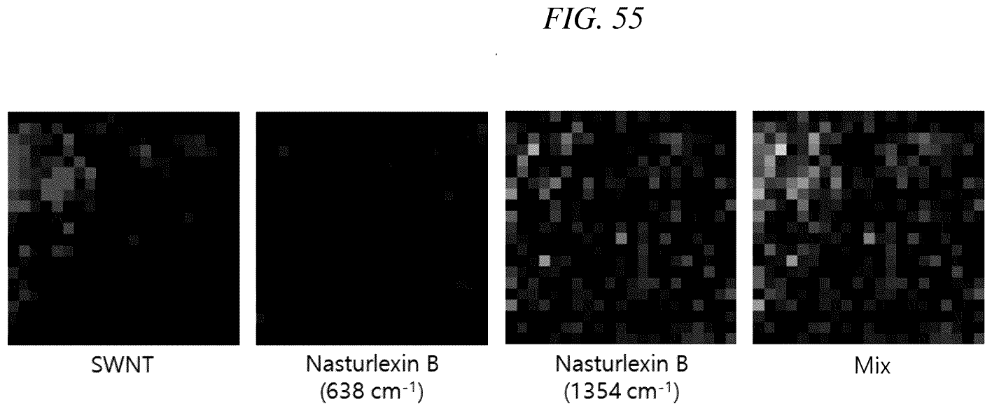

FIG. 55 is a SERS intensity map illustrating the results obtained by detecting a signal caused due to a wound in a watercress plant by using the SERS nanosensor according to the embodiment of FIG. 53. Here, the SERS signal was displayed as a color map. Red corresponds to SWNT, blue to nasturlexin B (638 cm⁻¹), and yellow to nasturlexin B (1354 cm⁻¹). The SERS spectrum was acquired by using a 785 nm laser at 3 mW.

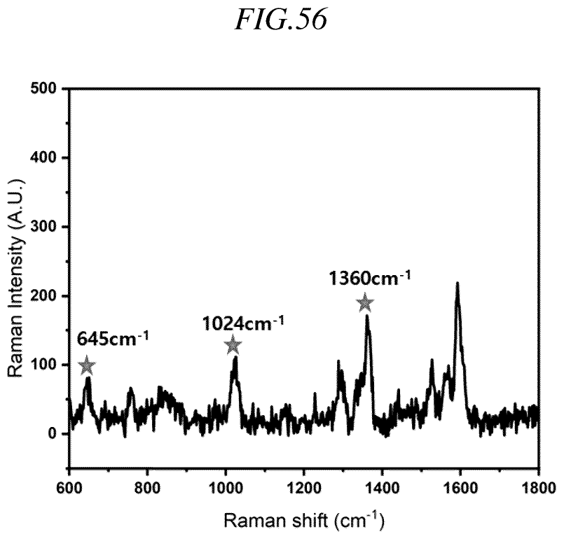

FIG. 56 is a graph illustrating raw SERS spectra for multiple molecular signals obtained at specific points of the SERS intensity map of FIG. 55.

Referring to FIG. 55 and FIG. 56, the SERS intensity map of a leaf with embedded nanosensors enables real-time monitoring of plant defense signal responses by visualizing simultaneous and multiple detection of various defense signal molecules in living plants.

According to the embodiments of the present invention described above, SERS nanosensors which may be usefully applied to the diagnosis of plant diseases or monitoring of plant conditions, and may easily detect substances produced within plants (i.e., substances produced in plants) may be implemented. Additionally, according to embodiments of the present invention, a plant monitoring apparatus and method using the SERS nanosensor may be implemented.

According to embodiments of the present invention, core technological capabilities of early diagnosis of plant diseases may be secured through the convergence of nanotechnology (NT) and biotechnology (BT). All technologies and platforms related to nanosensors according to embodiments of the present invention may be used to develop nanosensors (nano-optical sensors) for early diagnosis of various crop diseases. The plant diagnosis technology using the nanosensor described above may be employed usefully in developing reliable disease response measures through accurate and rapid initial diagnosis before lesions occur in actual agricultural industry sites.

In addition, early diagnosis of plant diseases using the above-mentioned nanosensor is a simple, non-destructive method which is easy to apply to various plant species, and may be a real-time detection method which may detect signals immediately after introducing the nanosensor into the plant. Therefore, it is expected that the early diagnosis of plant diseases (i.e., a plant monitoring method) using the nanosensor according to the embodiment may be commercialized as a form of a platform considering user convenience.

Additionally, embodiments of the present invention may be applied to precision agriculture, smart agriculture, new crop screening technology, plant biotechnology-based pharmaceutical production, etc.

The expected effects of the technology according to the embodiments of the present invention may be summarized from technological and economic and industrial aspects as follows.

### Expected Effects From a Technological Aspect

\textcircled{1} Advancement of early diagnosis method development technology for plant diseases and expansion of research fields.

\textcircled{2} Securing source technology for early diagnosis of plant diseases using nanotechnology.

\textcircled{3} Contribution to standardizing early and precise diagnosis of plant fungal diseases using nanosensors.

\textcircled{4} Providing new and more efficient technologies for producing organic products.

### Expected Effects in Economic and Industrial Aspects

\textcircled{1} Through early diagnosis using nanosensors, damage to farms is reduced by preventing and alleviating crop diseases which are problematic not only during the growing period but also after harvest and for which pesticides are less effective.

\textcircled{2} Expect the development of related industries through popularization of plant disease diagnosis technology using nanosensors and vitalization of research.

\textcircled{3} Revitalizing the domestic eco-friendly agricultural industry and promoting agricultural exports.

In this specification, the preferred embodiments of the present invention have been disclosed, and although specific terms have been used, they are only used in a general sense to easily explain the technological content of the present invention and to help understanding the present invention, and they are not used to limit the scope of the present invention. It is obvious to those having ordinary skill in the related art to which the present invention belong that other modifications based on the technological idea of the present invention may be implemented in addition to the embodiments disclosed herein. It will be understood to those having ordinary skill in the related art that in connection with SERS nanosensor for detecting a substance produced in a plant and its manufacturing method, and plant monitoring apparatus and method using the SERS nanosensor according to the embodiments described with reference to FIGS. 1 to 56, various substitutions, changes, and modifications may be made without departing from the technological spirit of the present invention. Therefore, the scope of the invention should not be determined by the described embodiments, but should be determined by the technological concepts described in the claims.

### INDUSTRIAL APPLICABILITY

The embodiments of the present invention may be applied to precision agriculture, smart agriculture, new crop screening technology, plant biotechnology-based pharmaceutical production, etc.

1. A surface-enhanced Raman scattering (SERS) nanosensor for detecting a substance produced within a plant comprising:
a first nanostructure;
a second nanostructure containing a metal and disposed on a surface of the first nanostructure to cause SERS; and
a polymer material bound to a surface of the second nanostructure, and generating an attraction force that attracts the substance produced within the plant.

2. The SERS nanosensor for detecting a substance produced within a plant of claim 1, wherein the first nanostructure includes a non-metal.

3. The SERS nanosensor for detecting a substance produced within a plant of claim 1, wherein the first nanostructure has a shape of a nanoparticle or a nanotube.

4. The SERS nanosensor for detecting a substance produced within a plant of claim 1, wherein the first nanostructure includes a silica or a carbon nanotube (CNT).

5. The SERS nanosensor for detecting a substance produced within a plant of claim 1, wherein the second nanostructure includes a plurality of nanoparticles.

6. The SERS nanosensor for detecting a substance produced within a plant of claim 1, wherein the second nanostructure includes at least one of Ag and Au.

7. The SERS nanosensor for detecting a substance produced within a plant of claim 1,
wherein the first nanostructure includes a silica nanoparticle,
wherein the second nanostructure includes a plurality of Ag nanoparticles disposed on a surface of the silica nanoparticle,
wherein the silica nanoparticle constitutes a core portion, and the plurality of Ag nanoparticles constitute a shell portion.

8. The SERS nanosensor for detecting a substance produced within a plant of claim 1,
wherein the first nanostructure includes a CNT,
wherein the second nanostructure includes a plurality of Au nanoparticles disposed on a surface of the CNT.

9. The SERS nanosensor for detecting a substance produced within a plant of claim 1, wherein the polymer material includes PDDA [poly(diallyldimethylammonium chloride)].

10. The SERS nanosensor for detecting a substance produced within a plant of claim 1, wherein the substance produced within the plant includes a plant hormone molecule generated by plant stress or disease.

11. The SERS nanosensor for detecting a substance produced within a plant of claim 1, wherein the substance produced within the plant includes at least one of phytoalexin, salicylic acid (SA), adenosine triphosphate (ATP), indole-3-acetic acid (IAA), folic acid (FA), thiamine, and nasturlexin.

12. A plant monitoring apparatus comprising:
a SERS nanosensor for detecting a substance produced within a plant according to claim 1; and
a Raman spectrometer for detecting a SERS signal generated from the SERS nanosensor.

13. A plant monitoring method comprising:
introducing a SERS nanosensor for detecting a substance produced within a plant according to claim 1 into a plant; and
measuring a SERS signal generated from the SERS nanosensor by using Raman spectroscopy.

14. A manufacturing method of a surface-enhanced Raman scattering (SERS) nanosensor for detecting a substance produced within a plant comprising:
preparing a first nanostructure;
forming a second nanostructure which is disposed on a surface of the first nanostructure, contains a metal and causes SERS; and
binding a polymer material which generates an attraction force that attracts the substance produced within the plant to a surface of the second nanostructure.

15. The manufacturing method of a SERS nanosensor for detecting a substance produced within a plant of claim 14,
wherein the first nanostructure includes a silica nanoparticle,
wherein the second nanostructure includes a plurality of Ag nanoparticles disposed on a surface of the silica nanoparticle,
wherein the silica nanoparticle constitutes a core portion,
wherein the plurality of Ag nanoparticles constitute a shell portion.

16. The manufacturing method of a SERS nanosensor for detecting a substance produced within a plant of claim 15, comprising:
functionalizing the surface of the silica nanoparticle with a thiol group by using 3-mercaptopropyltrimethoxysilane;
forming the plurality of Ag nanoparticles on the surface of the silica nanoparticle by using hexadecylamine and silver nitrate; and
functionalizing the surface of the plurality of Ag nanoparticles with the polymer material.

17. The manufacturing method of a SERS nanosensor for detecting a substance produced within a plant of claim 14,
wherein the first nanostructure includes a carbon nanotube (CNT),
wherein the second nanostructure include a plurality of Au nanoparticles disposed on a surface of the CNT.

18. The manufacturing method of a SERS nanosensor for detecting a substance produced within a plant of claim 14, wherein the polymer material includes PDDA [poly(diallyldimethylammonium chloride)].

19. The manufacturing method of a SERS nanosensor for detecting a substance produced within a plant of claim 14, wherein the first nanostructure has a shape of a nanoparticle or a nanotube.

20. The manufacturing method of a SERS nanosensor for detecting a substance produced within a plant of claim 14, wherein the second nanostructure includes a plurality of nanoparticles.
